(19) United States
(12) Patent Application Publication
(10) Pub. No.: US 2023/0201487 A1
(43) Pub. Date: Jun. 29, 2023

(54) NEBULIZER DEVICE OPTIMIZATION FOR IMPROVED AEROSOL PARAMETERS AND USES THEREOF

(71) Applicant: Avalyn Pharma Inc., Seattle, WA (US)

(72) Inventors: Mark William Surber, San Diego, CA (US); Stephen Pham, San Diego, CA (US)

(21) Appl. No.: 18/171,930

(22) Filed: Feb. 21, 2023

Related U.S. Application Data
(63) Continuation of application No. PCT/US2021/051598, filed on Sep. 22, 2021.
(60) Provisional application No. 63/081,735, filed on Sep. 22, 2020.

(51) Int. Cl. A61M 11/00; A61M 15/00; A61K 9/08; A61K 9/00; A61K 31/4418

(52) U.S. Cl. CPC A61M 11/005; A61M 15/0021; A61M 15/0086; A61M 15/0013; A61K 9/08; A61K 9/0078; A61K 31/4418

(57) ABSTRACT

Disclosed herein is a nebulizer comprising of a medicine cup reservoir containing an aqueous pirfenidone solution a medicine cup reservoir cap, an aerosol generator, an aerosol mixing chamber to which freshly generated aerosol resides until inhaled, a one-way inhalation valve, a mouthpiece and a one-way exhalation valve. The invention allows atmospheric pressure to be maintained inside the medicine cup reservoir during nebulization and optimizes the volume of the aerosol mixing chamber to minimize freshly generated aerosol inter-droplet collision, impaction of aerosol to the aerosol mixing chamber wall, droplet growth and/or condensation during exhalation, prior to inhalation, or during inhalation. The larger aerosol mixing chamber volume also allows the aerosol to accumulate during the exhalation phase. Despite venting producing a larger generated aerosol droplet population mean compared to the non-vented aerosol generator, The combined effect of the invention increases device output rate of respirable aerosol droplets, increases pirfenidone Cmax and AUC to improve treatment or prevention of various diseases, including disease associated with the lung, heart and kidney, including fibrosis, inflammatory conditions and transplant rejection.

### BACKGROUND OF THE INVENTION

Liquid nebulization of solutions containing active pharmaceutical ingredients has many advantages for delivering medications to the lung, by example large dose volume, large respirable dose, and immediately bioavailable delivered dose. However, the performance criteria vary broadly across dozens of nebulizer device mechanisms and constructs. Moreover, the performance of any particular Active Pharmaceutical Ingredient (API) and formulation can vary depending on the design and performance criteria of the nebulizer.

Moreover, each active pharmaceutical ingredient (API) behaves differently when an aqueous solution containing the API is converted into an aerosol by the nebulizer. Different, and unpredictable, physicochemical properties inherent to the API and formulation dictate the device and delivery parameters that enable delivery of a therapeutically effective dose of the API as an aerosol. For this reason, every new attempt to deliver an API by nebulization into an aerosol requires overcoming unforeseeable challenges that are encountered during drug and device development. This means that the selection of a nebulizer device for one medication may not hold for a different medication based on differences in the design and performance of a nebulizer that cannot be predicted and, if the wrong nebulizer is used, the design of the device may not be adequate to deliver a therapeutically effective dose.

In the absence of the ability to generate a therapeutically effective dose of an aerosol of a particular API, the pharmacodynamic profile of the API may render the API useless as an aerosol and this challenge requires the development of specific conditions and characteristics of all of the aqueous solution placed into a nebulizer, the operation of the nebulizer device to create the therapeutically effective aerosol, and the construction of the device that may be dictated by the unique characteristics of the API molecule dissolved in solution when it is converted to an aerosol.

### SUMMARY OF INVENTION

Described herein are nebulizer device designs that are specifically tailored to pharmaceutical formulations of pirfenidone (5-methyl-1 phenyl-2-1(H)-pyridone or 5-methyl-1-phenyl-2-(1H)-pyridone) dissolved in an aqueous solution containing other chemical elements to make aerosol compositions generated in the nebulizer described below stable and tolerable upon inhalation. The invention includes pirfenidone solutions containing other active ingredients, aerosol particles formed from the pharmaceutical formulations contained within the specially designed nebulizer, specific nebulizer device designs and methods for foregoing to selectively and favorably increase the ability to deliver a therapeutic dose of pirfenidone. Specifically, the API formulation and device are tailored to pharmacodynamic model that optimizes an aerosolized output rate to maximize the respirable dose to the patient. When an API is delivered to the lungs, an effective lung dose requires accumulation of the API in the lung tissue as delivered by aerosol and the effectiveness of this respirable delivered dose decreases over time as natural metabolic functions in the body eliminate the drug as it circulates systemically.

This natural clearance of the aerosol dose delivered to the lungs amplifies the importance of performance modifications in the nebulizer that increase respirable doses and dose delivery rates. This is particularly important where the API follows a Cmax “concentration maximum” pharmacodynamic profile, where a maximum short-lived peak dose is important, rather than an AUC “area under curve” model where the total quantity of delivered drug is important. Because the pharmacological effectiveness of pirfenidone is Cmax dependent, improvements of the respirable dose parameters by improving the nebulizer design and performance increases the therapeutic value of pirfenidone aerosols. Other APIs displaying the Cmax profile may also benefit from improvements in the respirable dose using the device parameters described below where elevated tissue concentrations of the API are desired by optimized delivery to the target tissue or compartment.

The present invention includes a nebulizer and nebulizer assembly that is specifically designed to have a medicine cup reservoir that contains liquid and to which an aqueous pirfenidone API solution is added prior to activating the aerosol-generating capability of the nebulizer device. The nebulizer device also preferably includes a medicine cup reservoir sealing structure to the contain the reservoir, an aerosol generator to create an aerosol of the pirfenidone API solution, an aerosol mixing chamber having a defined internal volume in which freshly generated aerosol resides until inhaled, a one-way inhalation valve, a mouthpiece, and a one-way exhalation valve. The aerosol generator may also operate in response to a breath-actuated circuit that triggers generation of the aerosol upon inhalation by a patient and may not include a dedicated aerosol mixing chamber of a defined size as described below.

In either of these embodiments a pirfenidone solution is disposed in a medicine cup reservoir that when used as directed is preferably sealed against leakage of the therapeutically effective pirfenidone dose within the medicine cup reservoir, although liquid sealed, a vent pathway engineered into the nebulizer when in operation allows atmospheric pressure to be maintained inside the medicine cup reservoir after addition of the pirfenidone solution to be nebulized and during aerosolization of the aqueous pirfenidone solution. The configuration of the medicine cup reservoir vent pathway for maintaining atmospheric pressure can be achieved by several different design approaches as described below that maintain atmospheric pressure throughout the entire administration delivery path of the API, from the solution disposed as a liquid in the medicine cup reservoir, through the aerosol generator, and optional aerosol mixing chamber, to establish a nebulization pathway that is unimpeded and maintained at ambient pressure from the liquid reservoir to the patient to optimize the parameters for the respirable delivered dose of pirfenidone.

In addition, the nebulizer aerosol mixing chamber volume has been optimized to define pressure and volume parameters that minimize freshly generated aerosol droplet collision, droplet growth and/or condensation during exhalation, impaction of the aerosol chamber wall prior to inhalation, or during inhalation from the aerosol chamber. The combined effect of these features on pirfenidone formulation administration is an increased device output rate of respirable aerosol droplets (amount of droplets less than 5 microns in diameter emitted from the device per unit time; respirable dose output rate). When an inhaled dose of pirfenidone is passed through the device as described below, the inhaled dose is both greater in aerosol concentration and also enhanced in terms of aerodynamic behavior of pirfenidone aerosol droplets generated Using this drug-device combination, these physiologically relevant parameters including, such as increased delivered drug Cmax and AUC are altered to improve treatment or prevention of various diseases, including disease associated with the lung, heart and kidney, including fibrosis, inflammatory conditions, infectious diseases, and transplant rejection.

For ease of reference when referring to the structure and function of the nebulizer, the portion of the nebulizer containing the medicine cup reservoir and the aqueous formulation of the API, and separated by the membrane of the aerosol generator, may be referred to as the “liquid side.” On the opposite side of the aerosol generator, and containing the air passage through which the aerosol passes from the aerosol generator to the patient, may be referred to as the “aerosol side.” The nebulizer may also be described as a “nebulizer assembly” when a separate vented container holding the aqueous API is inserted into the medicine cup reservoir to provide a separate, dedicated vent incorporated in the container that then becomes a part of the nebulizer assembly.

In one aspect of the invention, an improvement over the prior art for aerosol pirfenidone administration using an aqueous solution for nebulized administration comprising: water; pirfenidone or pyridone analog, including deuterated pirfenidone at a concentration from about 4.0-19.0 milligrams per milliliter with the permeant ion species and an osmolality-adjusting component, that may be the same chemical species, to yield a final solution in the device reservoir. The aqueous solution is contained and prepared for administration. In this configuration, the API exists simultaneously in different physical forms in the nebulizer: the liquid in the reservoir is maintained at ambient pressure to preserve the necessary nebulization parameters for a therapeutically effective pirfenidone API solution. The solution maintained at atmospheric pressure is directed to an aerosol generator that transforms the aqueous solution to an aerosol form have defined physical parameters resulting from the formulation and configuration of the nebulizer. The aerosol particles, in a defined concentration and particle distribution, are inhaled and at specified rates to provide the therapeutic dose.

To achieve this combination of effects, the aqueous pirfenidone solution has a series of improvements tailored to maximize the therapeutic potential of pirfenidone solutions delivered through the nebulizer described below, including one more inorganic salts selected from sodium chloride, magnesium chloride, calcium chloride, sodium bromide, magnesium bromide and calcium bromide in a concentration between 30 mM to about 450 mM. In some embodiments, the aqueous solution includes one more buffers selected from one or more of lysinate, glycine, acetylcysteine, phosphate, glutamate, acetate, borate, citrate, fumarate, malate, maleate, sulphate or Tris. In some embodiments, the pH of the aqueous solution is from about pH 3.0 to about pH 8.5. In some embodiments, the osmolality of the aqueous solution is from about 50 mOsmol/kg to about 1000 mOsmol/kg. In some embodiments, the buffer concentration in the aqueous solution is from about 0.01 mM to about 50 mM. In some embodiments, the solution further comprises one or more additional ingredients selected from tonicity agents, taste-masking agents, sweeteners, wetting agents, chelating agents, anti-oxidants, inorganic salts, and buffers. In some embodiments, the solution further comprises one or more additional ingredients selected from taste masking agents/sweeteners and inorganic salts. In some embodiments, the taste masking agent/sweetener is saccharin, or salt thereof. In some embodiments, described herein is a dose volume from about 0.5 mL to about 10 mL of the aqueous solution described herein.

In some embodiments, described herein is a kit comprising: a unit dosage of an aqueous solution of pirfenidone or pyridone analog, including deuterated pirfenidone as described herein in a container that is adapted for use in the featured nebulizer.

To maximize the therapeutic efficacy of inhaled pirfenidone species (including deuterated pirfenidone), the drug-device combinations of the present invention may increase the tissue target concentration contacted by the aerosol having the parameters defined below, achieving the unique aerosol composition and particle size distribution parameters in the aerosol mixing chamber downstream of, and distal along the administration vent pathway to, the nebulizer aerosol generator, wherein the aerosol mixing chamber has the defined dimensions, volume and pressure characteristics, and vented medication reservoir which yields shorter inhaled administration times while simultaneously enabling increased amounts and rates of delivered respirable drug.

Local delivery of inhaled pirfenidone will be cleared from lung tissue at a rate defined by the physicochemical characteristics of the pirfenidone molecule. Based on their respective physicochemical characteristics and associated pharmacodynamic profile, depending on the pirfenidone molecule and the specified pyridone analogs, some substances are more quickly eliminated from the pulmonary deposition location. To compensate, an increased delivery rate is required to out-compete local and systemic elimination and increase therapeutically effective concentrations of locally delivered drugs.

In one embodiment, pirfenidone or an analog thereof, whose delivered lung concentration correlates with activity, increasing the respirable dose delivery rate will bias the balance away from elimination to positively impact treatment or preventative effect; in effect, the faster a respirable dose is delivered, the greater the local Cmax and AUC. In some embodiments, the respirable dose delivery rate may be increased by increasing the number of aerosol droplets less than 5 microns that are generated in the nebulizer and traverse the volume of the aerosol chamber to be inhaled by a patient. In some embodiments, the respirable dose delivery rate may be increased by increasing the nebulizer output rate at which generated aerosol droplets having a preferred particle size and API concentration traverse the volume of the aerosol chamber to be inhaled by a patient. In some embodiments, the nebulizer output rate may be increased by using a medicine cup reservoir at ambient pressure with an aerosol generator disposed between the medicine cup reservoir and an aerosol mixing chamber also maintained at ambient pressure, through which generated aerosol droplets traverse the volume of the aerosol chamber to be inhaled by a patient. In some embodiments, the nebulizer output rate may be increased by using a medicine cup reservoir at ambient pressure with an aerosol generator disposed between the medicine cup reservoir and an aerosol mixing chamber also maintained at ambient pressure, wherein maintaining the number of generated aerosol droplets less than 5 microns in combination with an increased nebulizer output rate yields a greater quantity of respirable API per unit time that may be delivered to the patient through inhalation. In some embodiments, the respirable dose delivery rate may be increased by combining an increased number of droplets less than 5 microns and an increased nebulizer output rate.

In existing nebulizers, the act of loading the medication into the medicine cup reservoir and closing the medicine cup reservoir may create negative pressure inside the closed medicine cup reservoir. In these and other nebulizers, the action of nebulization of any API solution placed in the reservoir reduces the loaded dose volume in the closed medicine cup reservoir and creates negative pressure within that closed system. In such a case, negative pressure in the medicine cup reservoir slows the aerosol output rate and negatively impacts the resulting pharmacokinetics of delivered drug. This negative effect is further increased in cases where limited medicine cup reservoir dead volume exists prior to nebulization and where the output aerosol chamber has a limited internal volume. Typically, nebulizer device performance parameters are modelled on the use of a simple saline solution of dilute salt in water and the specific extent to which an API alters the performance of an aerosol formed from such a solution is unexpected and the ideal performance parameters remain to be determined for each API. AS described in the data presented below, pirfenidone in particular does not perform as expected relative to a saline standard.

To increase the nebulizer output rate and preserve desired aerosol particle size parameters, the pressure gradient created in the medicine cup reservoir during loading of the dosage form, closing the medicine cup reservoir and/or during the process of nebulization is minimized by maintaining ambient pressure inside the reservoir minimizing the pressure gradient across the aerosol generator, thereby providing an ambient pressure pathway from the reservoir through the aerosol generator and into the aerosol chamber from which the aerosol form of the nebulized solution is inhaled by the patient. The liquid nebulizer assembly has a medicine cup reservoir to which the medicine to be nebulized is added, a medicine cup reservoir cap, an aerosol generator, an aerosol mixing chamber, a one-way inhalation valve, a mouthpiece and a one-way exhalation valve wherein the entire system is maintained at ambient pressure through a series of venting structures comprised of vent pathways on the reservoir or liquid side and ports and valves on the aerosol side. In some embodiments, either of the medicine cup reservoir or medicine cup reservoir cap is vented to maintain atmospheric pressure inside the medicine cup reservoir after addition of the medicine to be nebulized and the cap installed. In some embodiments, atmospheric pressure is maintained by not installing the medicine cup reservoir cap onto the medication cup reservoir and relying on a separate mechanical expedient, such as a dedicated API delivery container mated to the opening of the medicine cup reservoir of the nebulizer to avoid spillage of the API and incorporating a venting pathway into the delivery container. In some embodiments, the medicine cup reservoir or medicine cup reservoir cap are structurally modified to maintain atmospheric pressure from the event of loading the medicine throughout dose nebulization and administration.

The respirable dose may be increased by generating smaller aerosol droplets. This may be accomplished through a variety of means including modified pressure in a jet nebulizer, optimizing the frequency of an ultrasonic nebulizer, changing the nozzle diameter and/or distance between the nozzle and the impinging surface of an impinging jet nebulizer, or conditioning the aerosols through a diffusion dryer, or perforated membrane hole size within a pressure-based or vibrating mesh aerosol generator.

The respirable dose may be increased by reducing the perforated membrane hole size within a mesh aerosol generator. However, reducing hole diameter may also reduce the nebulizer aerosol output rate. Alternatively, one can compensate by increasing the volume of the aerosol mixing chamber (device compartment holding freshly generated aerosol) to reduce aerosol inter-droplet collisions and impaction with the aerosol mixing chamber wall, droplet growth and/or condensation during the exhalation phase, prior to inhalation, or during inhalation. The larger volume of the aerosol mixing chamber also enables more continuously generated aerosol to accumulate during the exhalation phase. The liquid nebulizer mesh aerosol generator contains a small hole diameter in the perforated membrane, which generates aerosol droplets with a volume median diameter less than 5 microns.

The respirable dose output rate is increased by maintaining atmospheric pressure in the medicine cup reservoir throughout nebulized dose administration, including by providing a vent disposed in the body of the nebulizer, to increase the rate of the respirable delivered particles produced on the aerosol side of the aerosol generator.

The respirable dose output rate may be increased by reducing the perforated membrane hole size within a mesh aerosol generator in combination with maintaining atmospheric pressure in the medicine cup reservoir throughout nebulized dose administration, including by providing a vent disposed in the body of the liquid side of the nebulizer.

The act of increasing the respirable dose output rate by combining a small, perforated membrane hole size within a mesh aerosol generator and venting the medicine cup reservoir may increase the amount of larger particles, in effect increasing the population average aerosol droplet volumetric median diameter. Adding an increased volume aerosol mixing chamber to this configuration maintains the desired respirable delivered dose parameters within this increased population average aerosol droplet size in the quantity of aerosol maintained in the increased volume. In doing so, the number of respirable aerosol particles is maintained in the aerosol phase rather than condensing onto one another or impacting on an inner surface of the nebulizer or sedimentation onto the bottom of the aerosol chamber, contributing to an increased respirable dose output rate. In the present invention, the liquid nebulizer mesh aerosol generator contains thousands of small holes whose diameter is designed to generate aerosol droplets of an aqueous pirfenidone solution with a volumetric median diameter less than 5 microns and is coupled with a vented medicine cup reservoir and increased volume aerosol mixing chamber.

The liquid nebulizer mesh aerosol generator contains thousands of small holes whose diameter is designed to generate aerosol droplets with a volumetric median diameter less than 5 microns and is coupled with an increased volume aerosol mixing chamber and a vented medicine cup reservoir to maintain atmospheric pressure through the entire aerosol pathway comprising the medicine cup reservoir disposed within a vented nebulizer establishing atmospheric pressure on the solution side of the aerosol generating membrane, together with the increased volume aerosol mixing chamber and associated one-way valves for achieving the enhanced aerosol delivery parameters described below.

The liquid nebulizer mesh aerosol generator contains a small hole diameter generating aerosol droplets with a volumetric median diameter less than 5 microns and is coupled with an increased volume aerosol mixing chamber and a vented medicine cup reservoir to maintain atmospheric pressure throughout nebulized dose administration such that the development of negative pressure on the liquid side of the aerosol generator, within the liquid reservoir of the nebulizer, is avoided such that the liquid side pressure does not become negative or progressively more negative during the course of administration. As shown in the data below, this characteristic is important to maintain a consistent respirable delivered dose during the course of the administration and is a critical prerequisite to administering a therapeutic dose and obtaining the desired pharmacodynamic parameters in the lung, preferably within a defined set of parameters including time, volume, concentration of API, total dosage, and dosage rate parameters. Otherwise, the development of a negative or more negative pressure adversely impacts these parameters, especially the rate of drug delivery, and specifically the constancy of the rate of drug delivery that exhibits a negative slope over the duration of the administration of the unit dosage as negative pressure develops or increases in the liquid side of the nebulizer.

Knowing the pharmacodynamic profile of inhaled pirfenidone, particularly for the treatment of pulmonary fibrosis, maximizing the respirable drug that can be delivered in a limited time increases the therapeutic effect of the pirfenidone API when delivered by aerosol to maximize local dosage in the lung.

As noted above, generated pirfenidone aqueous solution aerosol characteristics are not as predicted when compared to the saline gold standard. Here, the combined effect of producing droplets with a volume median diameter less than 5 microns with an increased aerosol mixing chamber volume while maintaining medicine cup reservoir atmospheric pressure during nebulization of an aqueous pirfenidone solution nebulization, including deuterated pirfenidone liquid formulation, increases the respirable dose output and, upon inhalation, respirable dose delivery rate in such a way that a respirable therapeutic dose can be delivered within a time less than expected.

Increasing the aerosol mixing chamber volume reduces losses due to inter-droplet collisions and droplet impaction and sedimentation to the aerosol mixing chamber volume housing and allows aerosol to accumulate during an exhalation phase to reduce non-inhaled quantities of aerosol. Using the device parameters described below, an aqueous pirfenidone formulation is unexpectedly nebulized with a much higher output rate compared to saline solution with total solute contents remining similar so that calculated values for osmolality and other parameters can remain fixed.

Unexpectedly, maintaining atmospheric pressure in the medicine cup reservoir throughout nebulization to produce a larger average aerosol droplet population size acts synergistically in combination with the increased volume of the aerosol mixing chamber to maintain the amount of particles less than a 5 micron diameter even with an increased nebulization rate to effectively increase the device respirable dose output rate. The results presented in Example 1, Table 4, demonstrates that the structural and functional modifications to the nebulizer to maintain atmospheric pressure on the liquid side increases the respirable delivered dose per unit time between about 2% at the beginning of nebulization to about 21% by the end of nebulization. Separately, on the aerosol side, increasing the aerosol mixing chamber alone increases the respirable delivered dose per unit time by about 12%. Combining these two features synergistically increased the respirable delivered dose per unit time between about 15% at the beginning of nebulization to about 35% by the end of nebulization. This substantial respirable aerosol delivery rate increase benefits the concentration dependent pirfenidone activity by overcoming elimination to maximize pulmonary concentrations.

Achieving a beneficial drug concentration in the lung or downstream target tissue includes dependence upon two key factors: the rate at which inhaled droplets deposit in the lung and the rate at which drug within the deposited droplets eliminates from the lung. Increasing the nebulizer output rate while maintaining the respirable dose (amount of drug-containing aerosol droplets with a diameter less than 5 microns) allows deposited drug to bias the balance away from pulmonary elimination, permitting higher lung-deposited drug levels, and subsequent increased Cmax and AUC. This is of key importance for pirfenidone and pyridone analog, including deuterated pirfenidone whose mechanism is dependent on achieving high local drug concentrations.

The present invention also includes using the device parameters described herein to achieve a therapeutic concentration or quantity of pirfenidone or pyridone analog, pirfenidone or pyridone analog thereof selected from 1-Phenyl-2-(1H)pyridone, 5-methyl-1-(4-methylphenyl)-2-(1H)-pyridone, 5-Methyl-1-(2′-pyridyl)-2-(1H)pyridone, 6-Methyl-1-phenyl-3-(1H)pyridone, 6-Methyl-1-phenyl-2-(1H)pyridone, 5-Methyl-1-p-tolyl-3-(1H)pyridone, 5-Methyl-1-phenyl-3-(1H)pyridone, 5-Methyl-1-p-tolyl-2-(1H)pyridone, 5-Ethyl-1-phenyl-2-(1H)pyridone, 5-Ethyl-1-phenyl-3-(1H)pyridone, and 4-Methyl-1-phenyl-3-(1H)pyridone, and including deuterated forms for the foregoing.

Another benefit to the structural and functional device modifications described below is a reduction of the total time of nebulization and thus the time during which the patient must both activate the nebulizer and use a proper inhalation/breathing protocol to delivery of drug to have a therapeutic effect. In addition to the described pharmacokinetic benefits, the ability to deliver more drug to the middle and lower lung in less time, due to the increased respirable delivered dose rate, yields a shorter, more effective dosing regimen and increases patient compliance to nebulized dosing regimens. Overall, across a population of patients and varying compliance with nebulization protocols, therapeutic dose levels are achieved in more patients even with variations in compliance and a potential degradation in nebulizer device performance that can occur over time through repeated use of the nebulizer, including with sub-optimal cleaning regimens.

Improving the structural and functional performance of the nebulizer benefits the treatment or prevention various diseases, including interstitial lung disease (ILD), idiopathic pulmonary fibrosis (IPF), chronic fibrosing interstitial lung disease (CF-ILD), interstitial lung disease associated with systemic sclerosis (SSc-ILD), radiation-induced pulmonary fibrosis, viral-induced pulmonary fibrosis, COVID-19-induced pulmonary fibrosis, and other indications associated with progressive fibrosing interstitial lung disease (PFILD). The present invention also includes the treatment or prevention of chronic lung allograft dysfunction (CLAD) and bronchiolitis obliterans syndrome (BOS). The present invention also includes the treatment or prevention of inflammatory complications associated with viral infections (by non-limiting example COVID-19), asthma, and chronic obstructive pulmonary disease (COPD).

These device improvements also benefit the treatment or prevention of various heart diseases, including cardiac fibrosis, by example resulting from myocardial infarction, hypertensive heart disease, diabetic hypertrophic cardiomyopathy, idiopathic dilated cardiomyopathy, cardiac inflammatory conditions such as endocarditis, myocarditis, and pericarditis, and viral infections such as COVID-19.

These and other aspects of the invention will be evident upon reference to the following detailed description. All of the U.S. patents, U.S. patent application publications, U.S. patent applications, foreign patents, foreign patent applications and non-patent publications referred to in this specification, are incorporated herein by reference in their entirety, as if each was incorporated individually. Aspects of the invention can be modified, if necessary, to employ concepts of the various patents, applications and publications to provide yet further embodiments of the invention.

### CERTAIN TERMINOLOGY

The term “mg” refers to milligram.

The term “mcg” refers to microgram.

The term “microM” refers to micromolar.

The term “cc” refers to cubic centimeter.

The term “QD” refers to once a day dosing.

The term “BID” refers to twice a day dosing.

The term “TID” refers to three times a day dosing.

The term “QID” refers to four times a day dosing.

The term “Cmax” refers to the maximum concentration of a substance

The term “AUC” refers to the area under the time/concentration curve of a substance

The term “ELF” refers to lung epithelial lining fluid

As used herein, the term “about” is used synonymously with the term “approximately.” Illustratively, the use of the term “about” with regard to a certain therapeutically effective pharmaceutical dose indicates that values slightly outside the cited values, e.g., plus or minus 0.1% to 10%, which are also effective and safe.

The term “abnormal liver function” may manifest as abnormalities in levels of biomarkers of liver function, including alanine transaminase, aspartate transaminase, bilirubin, and/or alkaline phosphatase, and may be an indicator of drug-induced liver injury. See FDA Draft Guidance for Industry. Drug-Induced Liver Injury: Premarketing Clinical Evaluation, October 2007.

“Grade 2 liver function abnormalities” include elevations in alanine transaminase (ALT), aspartate transaminase (AST), alkaline phosphatase (ALP), or gamma-glutamyl transferase (GGT) greater than 2.5-times and less than or equal to 5-times the upper limit of normal (ULN). Grade 2 liver function abnormalities also include elevations of bilirubin levels greater than 1.5-times and less than or equal to 3-times the ULN.

A “therapeutic effect” relieves, to some extent, one or more of the symptoms associated with fibrosis, inflammation, or transplant rejection. This includes slowing the progression of, or preventing or reducing additional fibrosis, inflammation, or transplant rejection. For IPF and other forms of ILD and pulmonary fibrosis, a “therapeutic effect” is defined as a patient-reported improvement in quality of life and/or a statistically significant increase in or stabilization of exercise tolerance and associated blood-oxygen saturation, reduced decline in baseline forced vital capacity, decreased incidence in acute exacerbations, increase in progression-free survival, increased time-to-death or disease progression, and/or reduced lung fibrosis. For cardiac fibrosis, a “therapeutic effect” is defined as a patient-reported improvement in quality of life and/or a statistically significant improvement in cardiac function, reduced fibrosis, reduced cardiac stiffness, reduced or reversed valvular stenosis, reduced incidence of arrhythmias and/or reduced atrial or ventricular remodeling. For kidney fibrosis, a “therapeutic effect” is defined as a patient-reported improvement in quality of life and/or a statistically significant improvement in glomular filtration rate and associated markers. For disease resulting from active, previous or latent viral infection, a “therapeutic effect” is defined as a patient-reported improvement in quality of life and/or a statistically significant reduction in viral load, improved exercise capacity and associated blood-oxygen saturation, FEV1 and/or FVC, a slowed or halted progression in the same, progression-free survival, increased time-to-death or disease progression, and/or reduced incidence or acute exacerbation or reduction in neurologic symptoms. Need for treatment or prevention of chronic lung allograft dysfunction (CLAD), or lung transplant rejection, a “therapeutic effect” is defined as a patient-reported maintenance or improvement in quality of life and/or maintenance or increase in exercise tolerance and associated blood-oxygen saturation, reduced decline in baseline forced vital capacity, maintenance or reduced decline in forced expiratory volume of one second, maintenance or decreased incidence of acute exacerbations, maintenance or increased progression-free survival, maintenance or increased time-to-death or disease progression, and/or maintenance or reduced rate of progressive lung fibrosis, the latter measured by serial lung CT scans. For treatment or prevention of heart transplant rejection, a “therapeutic effect” is defined as a patient-reported maintenance or improvement in quality of life and/or maintenance or increase in ejection fraction. For treatment or prevention of kidney transplant rejection, a “therapeutic effect” is defined as a patient-reported maintenance or improvement in quality of life and/or maintenance or increase in, kidney creatinine or glomular filtration rate. “Treat”, “treatment”, or “treating”, as used herein refers to administering a pharmaceutical composition for therapeutic purposes. In some embodiments, the compositions described herein are used for prophylactic treatment. The term “prophylactic treatment” refers to treating a patient who is not yet diseased but who is susceptible to, or otherwise at risk of, a particular disease, or who is diseased but whose condition does not worsen while being treated with the pharmaceutical compositions described herein.

“Treat,” “treatment,” or “treating,” as used herein refers to administering a pharmaceutical composition for prophylactic and/or therapeutic purposes. The term “prophylactic treatment” refers to treating a patient who is not yet diseased, but who is susceptible to, or otherwise at risk of, a particular disease. The term “therapeutic treatment” refers to administering treatment to a patient already suffering from a disease. Thus, in preferred embodiments, treating is the administration to a mammal (either for therapeutic or prophylactic purposes) of therapeutically effective amounts of pirfenidone or pyridone analog, including deuterated pirfenidone.

The term “aerosol generator” refers to a nebulizer aerosol generation mechanism that converts an aqueous formulation of an API to a respirable aerosol dose.

The term “medicine cup reservoir” refers to the structural component on the liquid side of the nebulizer to which the medicine to be nebulized is added.

The term “medicine cup reservoir capacity” refers to the total volume of the medicine cup reservoir.

The term “aerosol mixing chamber” refers to the structural component on the aerosol side of the nebulizer having a housing containing an internal volume and that is down stream of the aerosol generator and to which newly generated aerosol resides until inhaled.

The term “L” in the context of the nebulizer aerosol mixing chamber refers to an aerosol mixing chamber with an internal volume of about 49 cubic centimeters, optionally in a vented embodiment of the nebulizer.

The term “XL” in the context of the nebulizer aerosol mixing chamber refers to an aerosol mixing chamber with an internal volume larger than the ‘L’ embodiment at incremental values of 10 cubic centimeters, of about 98 cubic centimeters, greater than about 98 cubic centimeters, greater than about 100, 110, 120, 130, 140 and cubic centimeters and as large as 150 cubic centimeters.

The term “dosing interval” refers to the time between administrations of the two sequential doses of a pharmaceutical during multiple dosing regimens.

The term “continuous daily dosing schedule” refers to the administration of the pyridone analog or pirfenidone every day at roughly the same time each day.

The term “respirable dose” is the amount of aerosolized pirfenidone or pyridone analog, including deuterated pirfenidone in aerosol droplets that are less than 5 microns in diameter.

The term “respirable delivered dose” (RDD) is the amount of aerosolized pirfenidone or pyridone analog, including deuterated pirfenidone in aerosol droplets less than 5 microns in diameter inhaled during the inspiratory phase.

The term “respirable dose delivery rate” is the amount of aerosolized pirfenidone or pyridone analog, including deuterated pirfenidone droplets less than 5 microns in diameter inhaled per unit time during the inspiratory phase.

The term “respirable dose output rate” is the amount of aerosolized droplets less than 5 microns in diameter emitted from the nebulizer per unit time.

The term “respirable fraction” is the percent of all generated aerosol droplets with a diameter less than 5 microns.

“Lung Deposition” as used herein, refers to the fraction of the nominal dose of an active pharmaceutical ingredient (API) that is deposited on the inner surface of the lungs.

### DESCRIPTION OF FIGURES

FIG. 1 is a prior art nebulizer exhibiting the basic structural elements for existing designs that deliver a nebulized aqueous solution to a patient by inhalation.

FIG. 2 is an exploded view of the improved nebulizer of the invention illustrating alternate approaches for establishing a vent pathway to maintain ambient pressure in a medicine cup reservoir during nebulization of an aerosol solution and the option for an expanded volume for an aerosol mixing chamber. FIG. 2A is a detailed view of the increased internal volume of an aerosol chamber in the greater than the L configuration.

FIG. 3 is an alternate view of the improved nebulizer of the invention showing the enlarged aerosol mixing chamber operably coupled to the medicine cup reservoir with the aerosol generator disposed therebetween. FIG. 3A is a detailed view of the increased internal volume of an aerosol chamber in the XL configuration.

FIG. 4 is a cross-section of the improved nebulizer of the invention showing the orientation of the headspace in the medicine cup reservoir, the aqueous solution contained in the medicine cup reservoir, the orientation of one embodiment of a medication cap, the internal volume of the XL aerosol mixing chamber proximate to the aerosol generator and the patient mouthpiece.

FIG. 5 is one embodiment of an ampoule or other container that is designed to fit within the medicine cup reservoir and having a vent pathway incorporated into the container itself rather than relying on a modification to the structure of the nebulizer as an alternate approach to maintain ambient pressure during aerosolization.

FIG. 6 is a schematic of an in-line version of the improved nebulizer of the invention incorporated into a forced-air ventilator respiratory circuit.

### DETAILED DESCRIPTION

### Pirfenidone, Pyridone Analogs and Deuterated Pirfenidone

As also noted elsewhere herein, in preferred embodiments the pyridone analog formulation as described herein comprises pirfenidone (5-Methyl-1-phenyl-2-(1H)-pyridone) or deuterated version or analogs thereof, including 1-Phenyl-2-(1H)pyridone, 5-methyl-1-(4-methylphenyl)-2-(1H)-pyridone, 5-Methyl-1-(2′-pyridyl)-2-(1H)pyridone, 6-Methyl-1-phenyl-3-(1H)pyridone, 6-Methyl-1-phenyl-2-(1H)pyridone, 5-Methyl-1-p-tolyl-3-(1H)pyridone, 5-Methyl-1-phenyl-3-(1H)pyridone, 5-Methyl-1-p-tolyl-2-(1H)pyridone, 5-Ethyl-1-phenyl-2-(1H)pyridone, 5-Ethyl-1-phenyl-3-(1H)pyridone, and 4-Methyl-1-phenyl-3-(1H)pyridone, and including deuterated forms for the foregoing.

### Pulmonary and Regional Diseases

A number of pulmonary diseases such as interstitial lung disease (ILD; and sub-class diseases therein), fibrotic indications of the lungs, kidney, heart, and inflammatory and fibrotic indications resulting from viral infections and other pathologies either idiopathic or attributed to specific molecular mechanisms are current areas of unmet clinical need due to the fact that either no particular pharmaceutical intervention as proved therapeutic or that different modes of administration of an API have proven ineffective or have exhibited such significant drawbacks, for example upon oral administration of pirfenidone, that the potential therapeutic value is not realized.

In fibrosis, scarring serves a valuable healing role following injury. However, tissue may become progressively scarred following more chronic and or repeated injuries resulting in abnormal function. In the case of idiopathic pulmonary fibrosis (IPF; and other subclasses of ILD, including chronic fibrosing ILD or the progressive phenotype and ILD associated with systemic sclerosis), if a sufficient proportion of the lung becomes scarred respiratory failure can occur. In any case, progressive scarring may result from a recurrent series of insults to different regions of the organ or a failure to halt the repair process after the injury has healed. In such cases the scarring process becomes uncontrolled and deregulated. In some forms of fibrosing disease scarring remains localized to a limited region, but in others it can affect a more diffuse and extensive area resulting in direct or associated organ failure.

In epithelial injury, epithelial cells are triggered to release several pro-inflammatory and pro-fibrotic mediators, including interleukin-1β, the potent fibroblast growth factors transforming growth factor-beta (TGF-beta), tumor necrosis factor (TNF), platelet derived growth factor (PDGF), endothelin, other cytokines, metalloproteinases and the coagulation mediator tissue factor. Importantly, the triggered epithelial cell becomes vulnerable to apoptosis, and together with an apparent inability to restore the epithelial cell layer are the most fundamental abnormalities in fibrotic disease.

In conditions such as diseases, physiological responses characterized by control of pro-inflammatory and pro-fibrotic factors with pyridone analog, such as pirfenidone may be beneficial to treat or prevent fibrosis, inflammation, or transplant rejection. Therapeutic strategies exploiting such pyridone analogs and/or pirfenidone effects in these and other indications are contemplated herein.

The mechanism of action for pyridone analogs, such as pirfenidone is to regulate production of cytokines and growth factors. These effects may directly result from direct pirfenidone exposure or may reflect secondary effects related to modulation of a single molecular target. In either event, pirfenidone modulation of cytokines, growth factors and markers of oxidative stress demonstrate that the anti-fibrotic effects observed in vivo are associated with regulation of pathways relevant to ongoing fibrosis and provide support for the observed anti-fibrotic effects.

For all of these diseases, and for the conditions described below, the improved aerosol delivery of API through enhanced respirable delivered dosages enabled by the improved nebulizer designs disclosed herein improves the therapeutic efficacy of the compound and the overall treatment of the disease.

### Interstitial Lung Disease, Pulmonary Fibrosis and Transplant Rejection

Interstitial lung disease (ILD) comprises and variety of fibrotic indications including by example idiopathic pulmonary fibrosis (IPF), chronic fibrosing ILD or the progressive phenotype and ILD associated with systemic sclerosis. These and other pulmonary fibrotic indications will be referred to herein as pulmonary fibrosis. Pulmonary fibrosis may be treated with a pyridone analog or pirfenidone. In some embodiments, the subject is mechanically ventilated. This group of disorders is characterized by scarring of deep lung tissue, leading to shortness of breath and loss of functional alveoli, thus limiting oxygen exchange. Etiologies include inhalation of inorganic and organic dusts, gases, fumes and vapors, use of medications, exposure to radiation, and development of disorders such as hypersensitivity pneumonitis, coal worker's pneumoconiosis, radiation, chemotherapy, transplant rejection, silicosis, byssinosis and genetic factors.

Exemplary fibrotic lung diseases for the treatment or prevention using the methods described herein include, but are not limited, idiopathic pulmonary fibrosis, chronic fibrosing ILD or the progressive phenotype, ILD associated with systemic schlerosis, pulmonary fibrosis secondary to systemic inflammatory disease such as rheumatoid arthritis, scleroderma, lupus, cryptogenic fibrosing alveolitis, radiation induced fibrosis, sarcoidosis, scleroderma, chronic asthma, silicosis, asbestos induced pulmonary or pleural fibrosis, acute lung injury and acute respiratory distress (including bacterial pneumonia induced, trauma induced, viral pneumonia induced, ventilator induced, non-pulmonary sepsis induced, and aspiration induced).

In some embodiments, the subject is a subject being mechanically ventilated and connected to an in-line nebulizer that operates according to the design parameters disclosed herein.

### Extrapulmonary Disease

A method for treating or preventing progression of an extrapulmonary disease, comprising administering a pyridone analog or pirfenidone to a middle to lower respiratory tract of a subject having or suspected of having extrapulmonary disease through oral inhalation of an aerosol comprising a pyridone analog or pirfenidone for purposes of pulmonary vascular absorption and delivery to extrapulmonary diseased tissues. In some embodiments, the extrapulmonary disease is cardiac fibrosis. The term “cardiac fibrosis” by non-limiting example relates to remodeling associated with or resulting from viral or bacterial infection, surgery, Duchenne muscular dystrophy, radiation therapy, chemotherapy, transplant rejection and chronic hypertension where myocyte hypertrophy as well as fibrosis is involved and an increased and non-uniform deposition of extracellular matrix proteins occurs. Fibrosis occurs in many models of hypertension leading to an increased diastolic stiffness, a reduction in cardiac function, an increased risk of arrhythmias and impaired cardiovascular function. In some embodiments, the extrapulmonary disease is heart transplant rejection. In some embodiments, the subject is a subject being mechanically ventilated.

A method for treating or preventing progression of an extrapulmonary disease, comprising administering a pyridone analog or pirfenidone to a middle to lower respiratory tract of a subject having or suspected of having extrapulmonary disease through oral inhalation of an aerosol comprising a pyridone analog or pirfenidone for purposes of pulmonary vascular absorption and delivery to extrapulmonary diseased tissues in improved dosages provided by the improvement in the structural and functional performance of the nebulizer as described herein are. In some embodiments, the extrapulmonary disease is kidney fibrosis. In some embodiments, the extrapulmonary disease is kidney transplant rejection. The term “kidney fibrosis” by non-limiting example relates to remodeling associated with or resulting chronic infection, obstruction of the ureter by calculi, malignant hypertension, radiation therapy, transplant rejection, severe diabetic conditions or chronic exposure to heavy metals. In some embodiments, kidney fibrosis correlates well with the overall loss of renal function. In some embodiments, the subject is a subject being mechanically ventilated.

### Liquid Nebulizer

The amount of drug that is placed in the nebulizer prior to administration to the mammal is generally referred to the “nominal dose,” or “loaded dose.” The volume of solution containing the nominal dose is referred to as the “fill volume.” Smaller droplet sizes or slow inhalation rates permit deep lung deposition. Both middle-lung and alveolar deposition may be desired for this invention depending on the indication, e.g., middle and/or alveolar deposition for pulmonary fibrosis and systemic delivery.

The improved nebulizer design of the invention is applicable to any sealed system in which a negative pressure develops on the liquid side of the device as an aqueous solution containing API is converted to aerosol. The potential nebulizer designs include ultrasonic nebulizers, pulsating membrane nebulizers, nebulizers with a vibrating mesh or plate with multiple apertures, non-vibrating mesh nebulizers (Omron Microair®), and nebulizers comprising a vibration generator and an aqueous chamber (e.g., PARI eFlow®). Commercially available nebulizers suitable for use in the present invention can include the Aeroneb®, MicroAir®, Aeroneb® Pro, and Aeroneb® Go, Aeroneb® Solo, Aeroneb® Solo/Idehaler combination, Aeroneb® Solo or Go Idehaler-Pocket® combination, Philips InnoSpire Go, eFlow and eFlow Rapid® (PARI, GmbH), Vectura FOX®, MicroAir® (Omron Healthcare, Inc.), Aerodose® (Aerogen, Inc, Mountain View, Calif.), Omron Elite® (Omron Healthcare, Inc.), Omron Microair® (Omron Healthcare, Inc.), Lumiscope® 6610, (The Lumiscope Company, Inc.), Airsep Mystique®, (AirSep Corporation), Aquatower® (Medical 02Industries America), I-neb produced by Philips, Inc.

Exemplary ultrasonic nebulizers suitable to provide delivery of a medicament as described herein can include UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Lumiscope's Ultrasonic Nebulizer, Medisana Ultrasonic Nebulizer, Microstat Ultrasonic Nebulizer. Other nebulizers for use herein include 5000 Electromagnetic Neb, 5001 Electromagnetic Neb 5002 Rotary Piston Neb, Lumineb I Piston Nebulizer 5500, Aeroneb Portable Nebulizer System, Aerodose Inhaler. Exemplary nebulizers comprising a vibrating mesh or plate with multiple apertures are described by R. Dhand in New Nebuliser Technology—Aerosol Generation by Using a Vibrating Mesh or Plate with Multiple Apertures, Long-Term Healthcare Strategies 2003, (July 2003), p. 1-4 and Respiratory Care, 47: 1406-1416 (2002), the entire disclosure of each of which is hereby incorporated by reference.

Additional nebulizers suitable for use in the presently described invention include nebulizers comprising a vibration generator and an aqueous chamber. Such nebulizers are sold commercially as, e.g., PARI eFlow, and are described in U.S. Pat. Nos. 8,511,581, 7,458,372, 9,061,303, 8,387,895, 9,168,556, 6,983,747 6,962,151, 5,518,179, 5,261,601, and 5,152,456, 7,316,067 and US Publication numbers 2016/0310681, 2018/0221906 each of which is specifically incorporated by reference herein. Other marketed vibrating mesh devices include the Breelib™ breath activated vibrating mesh nebulizer from Vectura, Deepro™ from HCmed, Fox® vibrating mesh nebulizer, Akita® adaptations of the PART eFlow, NBM-2 from Simzo, the Air Pro series, AeroCentre series, AeroGo series, and Airkid® series nebulizers from Feellife, Microlife's NEB-800, Honsun's NB-810B, Apex's Mobi Mesh, Salivia's M-Neb Flow+, Prodigy's Mini-Mist®, Health&Life's HL100A, KTMed's Neplus (NE-SM1), B.Well's WN-114, DigiO2's Digio2®, Babybelle's BBU01, PARI's Velox, TaiDoc's TD-7001, K-jump′ KN-9100, Medpack's NE-SM1 and OK Biotech's DocSpray handheld vibrating mesh nebulizers. Investigational devices include Aerami's Afina (Philips, and product concept stage devices), MICRONICE™ from Tekceleo.

High efficiency liquid nebulizers are inhalation devices that are adapted to deliver a large fraction of a loaded dose to a patient. Some high efficiency liquid nebulizers utilize microperforated membranes as the aerosol generator. In some embodiments, the high efficiency liquid nebulizer also utilizes one or more actively or passively vibrating microperforated membranes as the aerosol generator. In some embodiments, the high efficiency liquid nebulizer contains one or more oscillating or pulsating membranes as the aerosol generator. In some embodiments, the high efficiency liquid nebulizer contains a vibrating mesh or plate with multiple apertures and optionally a vibration generator with an aerosol mixing chamber. In some such embodiments, the aerosol mixing chamber functions to collect (or stage) the aerosol from the aerosol generator. In some embodiments, a one-way inhalation valve is also used to allow an inflow of ancillary ambient air into the aerosol mixing chamber during an inhalation phase and is closed to prevent escape of the aerosol from the aerosol mixing chamber during an exhalation phase.

A one-way inhalation valve or vent pathway that opens the aerosol side of the nebulizer to ambient air may be placed in the housing of the aerosol mixing chamber or proximate to the liquid side of the device with a dedicated pathway from the vent path opening to the aerosol mixing chamber, see, e.g., U.S. Pat. No. 8,387,895.

A one-way exhalation valve is arranged in or near the mouthpiece which is mounted on the outlet of the aerosol mixing chamber and through which the patient inhales the aerosol from the aerosol mixing chamber. In some embodiments, the high efficiency liquid nebulizer is continuously operating and may be controlled by a patient actuated circuit initiating and/or terminating operation of the aerosol generator. In some embodiments, the high efficiency liquid nebulizer operation is breath actuated.

In some embodiments, the high efficiency liquid nebulizer contains a vibrating microperforated membrane of tapered nozzles against a bulk liquid will generate a plume of droplets without the need for compressed gas. In these embodiments, a solution in the microperforated membrane nebulizer is present within a medicine cup reservoir allowing contact with the aerosol generating membrane, the opposite side of which is open to air. The membrane is perforated by a large number of microscopic nozzle orifices. An aerosol is created when alternating acoustic pressure in the solution is built up in the vicinity of the membrane causing the fluid on the liquid side of the membrane to be emitted through the nozzles as uniformly sized droplets.

Some embodiments the high efficiency liquid nebulizers use passive nozzle membranes and a separate piezoelectric transducer that are in contact with the solution present within the medicine cup reservoir. In contrast, some high efficiency liquid nebulizers employ an active nozzle membrane, which use the acoustic pressure in the nebulizer to generate very fine droplets of solution via the high frequency vibration of the nozzle membrane.

Some high efficiency liquid nebulizers contain a resonant system. In some such high efficiency liquid nebulizers, the membrane is driven by a frequency for which the amplitude of the vibrational movement at the center of the membrane is particularly large, resulting in a focused acoustic pressure in the vicinity of the nozzle; the resonant frequency may be about 100 kHz. A flexible mounting is used to keep unwanted loss of vibrational energy to the mechanical surroundings of the atomizing head to a minimum. In some embodiments, the vibrating membrane of the high efficiency liquid nebulizer may be made of a nickel-palladium alloy by electroforming.

In some embodiments, the high efficiency liquid nebulizer (i) achieves lung deposition of at least about 30%, at least about 35%, at least about 40%, based on the nominal dose of a pyridone analog or pirfenidone compound administered to the mammal.

In some embodiments, the high efficiency liquid nebulizer (ii) provides a Geometric Standard Deviation (GSD) of emitted droplet size distribution of the solution administered with the high efficiency liquid nebulizer of about 1.0 to about 2.5, about 1.2 to about 2.5, about 1.3 to about 2.0, at least about 1.4 to about 1.9, at least about 1.5 to about 1.9, about 1.5, about 1.7, or about 1.9.

In some embodiments, the high efficiency liquid nebulizer (iii) provides a mass median aerodynamic diameter (MMAD) of droplet size of the solution emitted with the high efficiency liquid nebulizer of less than about 5 about 1 to about 5 In some embodiments, the high efficiency liquid nebulizer (iii) provides a volume median diameter (VMD) of less than about 5 μm, about 3 μm to about 5 μm. In some embodiments, the high efficiency liquid nebulizer (iii) provides a volume median diameter (VMD) of less than about 5 μm, about 3 to about 5 μm.

In some embodiments, the high efficiency liquid nebulizer (iv) provides a fine droplet fraction (FPF=%<5 microns) of aerosol droplets emitted from the high efficiency nebulizer of at least about 45% and as much as 75%.

| Aerosol Attribute | PARI data (min-max)^{a} | PPD data (min-max)^{b} |
|---|---|---|
| DD (%) | 54.2-66.2 | 51.2-63.7 |
| FPF (%) | 52.3-57.3 | 47.4-56.9 |
| RD (%) | 29.9-37.6 | 26.1-34.4 |

^{a}Data from laser diffraction
^{b}Data from cascade impaction

In some embodiments, the high efficiency liquid nebulizer (v) provides a volume output rate of at least 0.38 mL/min. In some embodiments, the high efficiency liquid nebulizer (vi) delivers at least about 50% of the fill volume to the mammal.

In some embodiments, the high efficiency liquid nebulizer provides an RDD of at least about 22% of the nominal dose and provides a total daily dose of pirfenidone greater than 25 mg, through an administration schedule that may require multiple doses in a single day using at least 0.5 ml per loaded dose of pirfenidone at a concentration greater than 4 mg/ml and preferably less than 19 mg/ml at a respirable delivered dose output rate greater than 2.8 mg/minute.

### Nebulizer Optimization

In sealed-reservoir nebulizers, the act of loading the medication into the medicine cup reservoir and closing the medicine cup reservoir creates a negative pressure inside the closed medicine cup reservoir—either when the cap is placed or as soon as the level of liquid in the reservoir is reduced. The conversion of the loaded dose volume of the aqueous API solution in in the closed medicine cup reservoir to aerosol creates an increasingly negative pressure within the closed system thereby created on the liquid side of the nebulizer and as defined by the inner volume of the reservoir and the barrier formed by the aerosol generator. In each case, negative pressure in the medicine cup reservoir slows the output rate and may negatively impact generated aerosol droplet size. This effect is further increased in existing nebulizer designs where limited medicine cup reservoir dead volume exists prior to nebulization.

The structure of the improved nebulizer comprises a medicine cup reservoir capable of containing a nominal loaded or fill dose containing a therapeutic dose of an API and a reserved headspace between the liquid volume of the aqueous formula of the API and the internal portion of the device housing, a medicine cup reservoir cap or enclosure formed from an API container, a vibrating mesh aerosol generator, a structural modification to the nebulizer to maintain ambient pressure in the reservoir by connecting the headspace of the reservoir to ambient pressure conditions, and optionally an aerosol mixing chamber to which freshly generated aerosol resides until inhaled, a one-way inhalation valve, a mouthpiece and a one-way exhalation valve. The structural modification that allows atmospheric pressure to be maintained inside the medicine cup reservoir after addition of the medicine to be nebulized has several structural options that all perform the function of establishing a vent path form the headspace of the reservoir to ambient conditions after the API dose is loaded and the reservoir operably sealed prior to operation of the nebulizer and during conversion of the solution to aerosol to yield the improved aerosol parameters as described herein. The medicine cup reservoir or medicine cup reservoir cap also allow a discrete step of maintaining medicine cup reservoir atmospheric pressure after dose loading, and throughout nebulization and dose administration. In addition, the nebulizer aerosol mixing chamber volume has been optimized to minimize freshly generated aerosol droplet collision, droplet growth and/or condensation and sedimentation during exhalation, prior to inhalation, or during inhalation. Unpredicted by saline, the individual effect of these features on pirfenidone formulation administration is an increased device output rate of respirable aerosol droplets less than 5 microns in diameter emitted from the device per unit time to increase the respirable dose delivery rate.

From human modeling, these features increase pirfenidone Cmax and AUC to improve treatment or prevention of various diseases, including disease associated with the lung, heart and kidney, including fibrosis, inflammatory conditions and transplant rejection where a minimum threshold of delivery of an aerosol of pirfenidone achieves a therapeutic result. Combining a therapeutically effective respirable dose delivery rate of a pirfenidone solution by nebulization, with the novel structural features of the nebulizer as described below, provides an additive effect based on synergy between a specially formulated pirfenidone solution for administration by aerosol and a performance output rate of respirable aerosol droplets criteria including having particle physical parameters idealized for therapeutic delivery of drug product.

### Nebulizer-Drug Combinations

In one aspect, the invention described herein is drug-device combination comprised of the improved nebulizer and the API formulated and packaged as a defined volume and concentration of the API such that a specific therapeutic dose of the aqueous solution results from use of the improved nebulizer with the solution for nebulized aerosol administration. In the prifenidone example, the aqueous solution comprises: water; pirfenidone or pyridone analog, including deuterated pirfenidone at a concentration from about 4.0-19.0 milligrams per milliliter in concentration with the permeant ion species and an osmolality-adjusting component, that may be the same species, to yield a final solution in the device reservoir. The aqueous pirfenidone solution also has a series of selected parameters tailored to maximize the therapeutic potential of pirfenidone solutions delivered through the improved nebulizer, including one more inorganic salts selected from sodium chloride, magnesium chloride, calcium chloride, sodium bromide, magnesium bromide and calcium bromide in a concentration between 30 mM to about 450 mM. In some embodiments, the aqueous solution includes one more buffers selected from one or more of lysinate, glycine, acetylcysteine, glutamine, acetate, borate, citrate, fumarate, malate, maleate, sulphate, phosphate or Tris. In some embodiments, the pH of the aqueous solution is from about pH 3.0 to about pH 8.5. In some embodiments, the osmolality of the aqueous solution is from about 50 mOsmol/kg to about 1000 mOsmol/kg. In some embodiments, the buffer concentration in the aqueous solution is from about 0.01 mM to about 50 mM. In some embodiments, the solution further comprises one or more additional ingredients selected from tonicity agents, taste-masking agents, sweeteners, wetting agents, chelating agents, anti-oxidants, inorganic salts, and buffers. In some embodiments, the solution further comprises one or more additional ingredients selected from taste masking agents/sweeteners and inorganic salts. In some embodiments, the taste masking agent/sweetener is saccharin, or salt thereof. In some embodiments, described herein is a dose volume from about 0.5 mL to about 10 mL of the aqueous solution described herein. In some embodiments, described herein has a pirfenidone aqueous solution concentration is about 4 mg/mL to about 19 mg/mL. In some embodiments, described herein is a device loaded aqueous solution contains 2 mg to about 152 mg pirfenidone. In some embodiments, described herein the about 2 mg to about 152 mg pirfenidone containing aqueous solution device loaded dose is delivered in less than 15 minutes. In some embodiments, described herein the about 2 mg to about 152 mg pirfenidone containing aqueous solution device loaded dose is delivered in less than 15 minutes, providing at least about 22 percent of the pirfenidone loaded dose in aerosol droplets less than 5 microns. In some embodiments, described herein about 6.25 mg to about 125 mg pirfenidone containing aqueous solution device loaded dose is delivered in less than 15 minutes, providing at least about 22 percent of the pirfenidone loaded dose in aerosol droplets less than 5 microns, that are in turn delivered this respirable delivered dose is delivered at a rate of at least 2.8 mg pirfenidone per minute.

In some embodiments, described herein is a kit comprising: a unit dosage of an aqueous solution of pirfenidone or pyridone analog, including deuterated pirfenidone as described herein in a container that is adapted for use in the improved nebulizer, and optionally containing the nebulizer with instructions for delivering the dose provided by the kit. Separately, the kit can provide specific instructions for use with the drug-device combination as part of a treatment regimen, including use, cleaning and/or maintenance instructions that are unique to the nebulizer described herein.

To maximize the efficacy of inhaled pirfenidone or pyridone analog, shorter inhaled administration times may be desired. Local delivery of an inhaled substance will be eliminated from its deposition site at a rate defined by its physicochemical characteristics and associated properties of the target tissue wherein the inhaled dose is deposited. As is the case with pirfenidone and pyridone analogs, some substances are eliminated quickly from the target tissue. To compensate, an increased delivery rate is required to out-compete elimination and increase the local concentration of the inhaled substance. More specifically, for pirfenidone and pyridone analogs, whose delivered concentration correlates with activity, increasing the respirable dose delivery rate (the rate at which inhaled droplets less than 5 microns in diameter are delivered to the target tissue) will bias the balance away from elimination to positively impact treatment or preventative effect; in effect, the faster a respirable dose is delivered, the greater the Cmax and AUC concentrations achieved at the target site. The respirable dose delivery rate may be increased by increasing the number of aerosol droplets less than 5 microns. In some embodiments, the respirable dose delivery rate may be increased by increasing the nebulizer output rate (increased aerosol production per unit time). In some embodiments, the respirable dose delivery rate may be increased by combining an increased number of droplets less than 5 microns and an increased nebulizer output rate.

In one embodiment, the respirable dose may be increased by reducing the perforated membrane hole size within a mesh aerosol generator. However, reducing hole diameter may also reduce the nebulizer aerosol output rate. Alternatively, one can compensate by increasing the volume of the aerosol mixing chamber to increase the quantity of the compartment holding freshly generated aerosol. The enlarged volume of the mixing chamber reduces aerosol inter-droplet collisions, droplet impaction of aerosol droplets to the wall of the aerosol mixing chamber and/or condensation of aerosol during the exhalation phase, prior to inhalation, or during inhalation. The larger internal volume also allows more aerosol to accumulate in the aerosol mixing chamber during the exhalation phase. In the present invention, the liquid nebulizer mesh aerosol generator contains thousands of small holes in a perforated membrane designed to generate aerosol droplets with a volume median diameter less than 5 microns.

In some embodiments, perforated membrane hole size within a mesh aerosol generator may be produced to generate an aerosol VMD that is more than about 3 microns and less than about 5 microns. In some embodiments, the medicine cup reservoir capacity is more than 4.0 ml, 6.0 ml, 8.0 ml and preferably less than 14 ml. The medicine cup reservoir dead volume after addition of a dosing solution is less than about 10 mL, less than about 8 mL, less than about 6 mL, less than about 4 mL, less than about 2 mL, less than about 1 mL, less than about 0.5 mL.

In some embodiments, the nebulizer may produce aerosol continuously. In other embodiments, the nebulizer aerosol production may be breath actuated. In some embodiments, the nebulizer may contain all components required for nebulization in a single unit. In other embodiments, the nebulizer may contain the components required for nebulization in more than one unit either connected by a wire or wirelessly, such as Bluetooth®.

Achieving a beneficial drug concentration in the lung or downstream target tissue is dependent on two key factors: the rate at which inhaled droplets deposit in the lung and the rate at which drug within the deposited droplets eliminates from the lung. Increasing the aerosol output rate while maintaining the respirable dose (amount of drug-containing aerosol droplets with a diameter less than 5 microns) allows deposited drug to bias the balance away from elimination, permitting higher deposited drug levels, and subsequent increased Cmax and AUC. This is of key importance for pirfenidone and pyridone analogs with a mechanism dependent on achieving increased local drug concentrations in the target tissue.

In some embodiments, pirfenidone compound formulation as disclosed herein, is placed in the preferred vibrating mesh nebulizer configuration and loaded with about 10 mg to about 100 mg pirfenidone in a dosing solution of about 0.5 mL to about 10 mL.

In some embodiments, each pyridone analog or pirfenidone respirable delivered dose is more than about 0.5 mg, more than about 4 mg, more than about 12.5 mg, more than about 22 mg, more than about 38 mg, more than about 50 mg. For a 4 mg/mL pirfenidone aqueous solution the respirable delivered dose is delivered at a rate more than about 0.9 mg/min. For a 12.5 mg/mL pirfenidone aqueous solution the respirable delivered dose is delivered at a rate more than about 2.8 mg/min. For a 19 mg/mL pirfenidone aqueous solution the respirable delivered dose is delivered at a rate more than about 4.3 mg/min.

In some embodiments, the pyridone analog or pirfenidone may be administered in the preferred vibrating mesh nebulizer configuration in less than about 25 min, less than about 20 min, less than about 18 min, less than about 16 min, less than about 14 min, less than about 12 min, less than about 10 min, less than about 8 min, less than about 6 min, less than about 4 min, less than about 2 min, less than about 1 min, in less than five breaths, in less than four breaths, in less than three breaths, in less than two breaths, or in one breath.

In some embodiments, the pyridone analog or pirfenidone may be administered in the preferred vibrating mesh nebulizer configuration to deliver lung epithelial lining fluid concentrations at more than 10 mcg/mL per minute, at more than 5 mcg/mL per minute, at more than 2.5 mcg/mL/minute.

In some embodiments, the pyridone analog or pirfenidone may be administered in the preferred vibrating mesh nebulizer configuration to deliver lung epithelial lining fluid exposures at more than 0.15 mg·hr/L per minute, at more than 0.10 mg·hr/L per minute, at more than 0.05 mg·hr/L per minute.

In one aspect, described herein is a method of achieving a lung epithelial lining fluid AUC₀₋₂₄ of a pyridone analog or pirfenidone, that is at least 1.1 times, at least 1.2 times, at least 1.3 times, at least 1.4 times, at least 1.5 times to at least 3 times the epithelial lining fluid AUC₀₋₂₄ resulting from delivery a pyridone analog or pirfenidone using an equivalent nebulizer loaded with the same dose, yet lacking the optimized features described herein. In one aspect, described herein is a method of achieving a lung epithelial lining fluid Cmax of a pyridone analog or pirfenidone, that is at least 1.1 times, at least 1.2 times, at least 1.3 times, at least 1.4 times, at least 1.5 times to about 3 times the epithelial lining fluid Cmax resulting from delivery a pyridone analog or pirfenidone using an equivalent nebulizer loaded with the same dose, yet lacking the optimized features described herein.

In some embodiments, continuous dosing schedule refers to the administration of the pyridone analog or pirfenidone at regular intervals without any drug holidays from the particular therapeutic agent. In some other embodiments, continuous dosing schedule refers to the administration of the pyridone analog or pirfenidone in alternating cycles of drug administration followed by a drug holiday (e.g., wash out period) from the pyridone analog or pirfenidone. For example, in some embodiments the pyridone analog or pirfenidone is administered once a day, twice a day, three times a day, once a week, twice a week, three times a week, four times a week, five times a week, six times a week, seven times a week, every other day, every third day, every fourth day, daily for a week followed by a week of no administration of the pyridone analog or pirfenidone, daily for a two weeks followed by one or two weeks of no administration of the pyridone analog or pirfenidone, daily for three weeks followed by one, two or three weeks of no administration of the pyridone analog or pirfenidone, daily for four weeks followed by one, two, three or four weeks of no administration of the pyridone analog or pirfenidone, weekly administration of the therapeutic agent followed by a week of no administration of the pyridone analog or pirfenidone, or biweekly administration of the therapeutic agent followed by two weeks of no administration of the pyridone analog or pirfenidone.

In some embodiments, the amount of repeat high Cmax dosing providing more regular exposure of the pyridone analog or pirfenidone that is given to the human varies depending upon factors such as, but not limited to, condition and severity of the disease or condition, and the identity (e.g., weight) of the human, and the pyridone analog or pirfenidone that are administered (if applicable).

All of the U.S. patents, U.S. patent application publications, U.S. patent applications, foreign patents, foreign patent applications and non-patent publications referred to in this specification are incorporated herein by reference, in their entirety.

### EXAMPLES

### Example 1. Nebulizer and Aerosol Performance Optimization

To measure the impact of aerosol mixing chamber volume (referred to herein as L and XL) and maintaining atmospheric pressure throughout nebulization and administration of the therapeutic dose to a patient using the nebulizer design described herein, as opposed to allowing increasing negative pressure to be generated as a negative pressure bias is developed in the pirfenidone reservoir as the volume of the reservoir headspace above the loaded dose volume increases over time during nebulization, the following data were assembled to measure and demonstrate the performance improvement.

As an initial analysis, emitted aerosol saline and pirfenidone aqueous solution formulation droplet size as a function of time was measured using a Helos, Sympatec laser under partially simulated breathing conditions (adult breathing pattern, 500 mL tidal volume, 15 breaths/min with a 1:1 inhalation: exhalation ratio) using a Compas 2 breath simulator. Briefly, twelve investigational PART eFlow® nebulizers (with 6 L and 6 XL aerosol mixing chambers) with a single set of 6 (six) aerosol head class 35 (6 total heads; Table 1) were used in both L (about 49 cc volume aerosol mixing chamber) and XL (about 98 cc volume aerosol mixing chamber) configurations and tested in triplicate with the 8 mL of pirfenidone aqueous solution medicine cup reservoir under atmospheric conditions (vented) and permitting generation of negative pressure (non-vented). The following exemplary replicates were performed: A. Head 1/L/vented, B. Head 1/L/non-vented, C. Head 1/XL/vented, D. Head 1/XL/non-vented, and repeated in the following order: A, B, C, D, A, B, C, D, A, B, C, D. Results are shown in Table 2.

TABLE 1
Saline - Aerosol head-only saline aerosol characteristics

| | 250 mbar below atmospheric | | | Atmospheric pressure | | |
|---|---|---|---|---|---|---|
| | pressure (non-vented reservoir) | | | (vented reservoir) | | |
| | VMD^{a} | | TOR^{c} | VMD | | TOR |
| No. | [μm] | GSD^{b} | [g/min] | [μm] | GSD | [g/min] |
| 1 | 3.55 | 1.48 | 0.901 | 3.91 | 1.57 | 1.205 |
| 2 | 3.46 | 1.48 | 0.904 | 3.76 | 1.56 | 1.018 |
| 3 | 3.52 | 1.49 | 0.905 | 3.83 | 1.58 | 1.101 |
| 4 | 3.87 | 1.53 | 1.100 | 4.22 | 1.61 | 1.309 |
| 5 | 3.42 | 1.46 | 0.650 | 4.06 | 1.53 | 0.478 |
| 6 | 3.43 | 1.46 | 0.750 | 3.64 | 1.54 | 0.379 |

^{a}VMD: Volume median diameter;
^{b}GSD: Geometric standard deviation;
TOR: Total output rate based upon an average output

In this analysis, respirable aerosol droplet output rate of saline and pirfenidone formulation was determined by multiplying the respirable fraction (RF; percent emitted aerosol droplets with a diameter less than 5 microns at 20 second increments) by total output rate (TOR; calculated by dividing the total nebulizer weight loss by nebulization duration). Results are shown in Tables 2 and 3. Saline was made as 150 mM sodium chloride in water, while the pirfenidone formulation was 12.5 mg/mL pirfenidone in 5 mM citrate buffer, pH 6.0, 150 mM sodium chloride and 0.75 mM sodium saccharin in water. Duration to nebulize 8 mL saline was 8.6 min, 12.6 min, 8.5 min and 12.0 min for vented “L”, non-vented “L”, vented “XL” and non-vented “XL”, respectively. Duration to nebulize 8 mL AP01 was 8.4 min, 12.3 min, 8.3 min and 12.4 min for vented “L”, non-vented “L”, vented “XL” and non-vented “XL”, respectively.

TABLE 2
Saline - Percent respirable aerosol droplet output rate benefit of moderating
medicine cup pressure, aerosol mixing chamber volume and the two features combined

| | | | | | | | Benefit^{c} (%) | | | | | |
|---|---|---|---|---|---|---|---|---|---|---|---|---|
| | | | | | | | | | Increased | | | |
| | | Respirable | | | | | Maintaining | | Aerosol | | Combined | |
| | | Aerosol Droplet | | | | | Atmospheric | | mixing | | Effect | |
| | | Output Rate^{b} | | | | | Pressure | | chamber | | (Vented + | |
| | Aerosol | (g/min) | | | | | (Vented) | | (XL) | | XL) | |
| Medicine Cup | mixing | 0-2 | 2-4 | 4-6 | 6-8 | | 0-2 | 6-8 | 0-2 | 6-8 | 0-2 | 6-8 |
| Configuration^{a} | chamber | min | min | min | min | Slope | min | min | min | min | min | min |
| V | L | 0.41 | 0.40 | 0.40 | 0.40 | −0.002 | −1.47^{d} | −6.40^{d} | 3.77^{e} | 4.56^{e} | | |
| NV | L | 0.41 | 0.42 | 0.42 | 0.42 | +0.002 | | | | | | |
| V | XL | 0.42 | 0.42 | 0.42 | 0.41 | −0.001 | −0.82^{f} | −5.69^{f} | 3.10^{g} | 3.77^{g} | 2.25^{h} | −2.13^{h} |

| | | | | | | |
|---|---|---|---|---|---|---|
| NV | XL | 0.43 | 0.43 | 0.44 | 0.44 | +0.002 |

^{a}V: vented (medicine cup maintained under atmospheric pressure), NV: non-vented (closed system medicine cup);
^{b}Respirable aerosol droplet output rate (gram aerosol droplets < 5 microns emitted per minute) during 2 minute nebulization increments;
^{c}Benefit measured as percent improvement between medicine cup pressure (vented vs. non-vented in either “L” or “XL” configurations), aerosol mixing chamber volume (“L” vs. “XL ” in either vented or non-vented configurations), and the combined benefit of a vented “XL” device configuration compared to a non-vented “L” device configuration.
^{d}vented “L” vs. non-vented “L”;
^{e}vented “L” vs. vented “XL”;
^{f}vented “XL” vs. non-vented “XL”;
^{g}non-vented “L” vs non-vented “XL”;
^{h}vented “XL” vs . non-vented “L”.

The saline data presented in Table 2 shows that venting the medicine cup reservoir while nebulizing saline has a small negative benefit on respirable aerosol droplet output rate (gram aerosol droplets <5 microns emitted per minute) during early administration (about −1.5% in the “L” configuration and about −1% in the “XL” configuration), and this negative effect increases slightly near the end of dosing (about −6.4% in the “L” configuration and about −5.5% in the “XL” configuration). The data also show that aerosol mixing chamber volume has a small positive benefit. Combining these two device elements shows an averaging effect, wherein the positive benefit observed for increasing aerosol mixing chamber volume mitigates the small negative effect associated with venting the medicine cup (about +2.3% in the early stage of saline dose administration, reducing to −2.1% in the latter stage). Based on this saline data, no justification would exist for modifying the housing of a nebulizer to affect the pressure profile over time or for modifying the size of the aerosol mixing chamber to improve the delivery parameters of an aerosol formed from an aqueous solution of pirfenidone, nor that any such modification could lead to a therapeutically effective result.

TABLE 3
Percent respirable aerosol droplet output rate benefit for aqueous
pirfenidone solution of moderating medicine cup pressure, aerosol
mixing chamber volume and the two features combined

| | | Respirable | | | | | Benefit^{c} (%) | | | | | |
|---|---|---|---|---|---|---|---|---|---|---|---|---|
| | | Aerosol | | | | | Maintaining | | Increased | | Combined | |
| | | Droplet | | | | | Atmospheric | | Aerosol mixing | | Effect | |
| | | Output Rate^{b} | | | | | Pressure | | chamber | | (Vented + | |
| | Aerosol | (g/min) | | | | | (Vented) | | (XL) | | XL) | |
| Medicine Cup | mixing | 0-2 | 2-4 | 4-6 | 6-8 | | 0-2 | 6-8 | 0-2 | 6-8 | 0-2 | 6-8 |
| Configuration^{a} | chamber | min | min | min | min | Slope | min | min | min | min | min | min |
| V | L | 0.43 | 0.42 | 0.42 | 0.42 | −0.0015 | 19.44^{d} | 17.51^{d} | −1.98^{e} | −4.19^{e} | | |
| NV | L | 0.36 | 0.36 | 0.36 | 0.36 | +0.0011 | | | | | | |
| V | XL | 0.42 | 0.42 | 0.42 | 0.40 | −0.0010 | 23.86^{f} | 16.99^{f} | −5.48^{g} | −3.77^{g} | 17.08^{h} | 12.58^{h} |

| | | | | | | |
|---|---|---|---|---|---|---|
| NV | XL | 0.34 | 0.34 | 0.34 | 0.34 | +0.0000 |

^{a}V: vented (medicine cup maintained under atmospheric pressure), NV: non-vented (closed system medicine cup);
^{b}Respirable aerosol droplet output rate (gram aerosol droplets < 5 microns emitted per minute) during 2 minute nebulization increments;
^{c}Benefit measured as percent improvement between medicine cup pressure (vented vs. non-vented in either “L” or “XL” configurations), aerosol mixing chamber volume (“L” vs. “XL” in either vented or non-vented configurations), and the combined benefit of a vented “XL” device configuration compared to a non-vented “L” device configuration,
^{d}vented “L” vs. non-vented “L”;
^{e}vented “L” vs. vented “XL”;
^{f}vented “XL” vs. non-vented “XL”;
^{g}non-vented “L” vs. non-vented “XL”;
^{h}vented “XL” vs. non-vented “L”.

Unlike that observed for saline, the data for a therapeutic aqueous solution of pirfenidone listed in Table 3 shows that venting the medicine cup reservoir has a strong positive benefit on respirable aerosol droplet output rate (about +19.4% in the “L” configuration and about +23.8% in the “XL” configuration), with a slight reduction near the end of dosing (to about +17.5% in the “L” configuration and about +17% in the “XL” configuration). Interestingly, aerosol mixing chamber volume exhibits a slight negative benefit on respirable aerosol droplet output rate (about −2 to −5.5% benefit in all device configurations regardless of medicine cup pressure). Combining these two device elements shows an averaging effect, wherein the positive effect of venting the medicine cup reservoir mitigates the small negative effect associated with increasing the medicine cup reservoir size (about +17% in the early stage of pirfenidone dose administration, reducing to +12.6% in the latter stages).

In these initial measurements, laser diffraction and gravimetric calculation were used to determine the amount of respirable aerosol droplets per unit time, the combined Table 2 and Table 3 data indicate that venting the nebulizer medicine cup reservoir has a strong positive benefit when nebulizing a therapeutic quantity of an aqueous pirfenidone solution and a negative benefit when nebulizing saline. Similarly, while increasing aerosol mixing chamber volume has a small negative effect on a therapeutic pirfenidone solution either venting the reservoir alone or venting the reservoir in combination with increasing aerosol mixing chamber volume increases the respirable aerosol droplet output rate of a therapeutic solution of pirfenidone much higher than would have been predicted by saline.

For a more clinically relevant comparison, in a second analysis, of aerosol delivery parameters for pirfenidone in solution, the respirable delivered dose (RDD; amount of deposited pirfenidone from aerosol droplets less than 5 microns in diameter) was measured during breath simulation (adult breathing pattern, 500 mL tidal volume, 15 breaths/min with a 1:1 inhalation: exhalation ratio) using a Compas 2 breath simulator was used as follows. Briefly, twelve investigational eFlow® (with 6 L and 6 XL aerosol mixing chambers) with a single set of 6 (six) aerosol head class 35 (6 total heads; Table 1) were used in both (about 49 cc volume) and XL (about 98 cc volume) aerosol mixing chamber configurations and tested in duplicate with the 8 mL medicine cup reservoir under atmospheric conditions (vented) and permitting generation of negative pressure (non-vented) during nebulization. An 8 mL dose (aqueous pirfenidone solution at concentration of 12.5 mg/ml) was loaded into the medicine cup reservoir and nebulization initiated. After 2, 4, 6 and 8 min of nebulization, inspiratory filters were collected and extracted for pirfenidone quantitation using HPLC analysis. A final filter was used after 8 min to collect remaining dose. The following exemplary replicates were performed: A. Head 1/L/vented, B. Head 1/L/non-vented, C. Head 1/XL/vented, D. Head 1/XL/non-vented, and repeated in the following order: A, B, C, D, A, B, C, D. Results are shown in Table 4.

TABLE 4
Percent pirfenidone respirable delivered dose benefit of moderating medicine cup
pressure, aerosol mixing chamber volume and the two features combined

| | | | | | | | Benefit^{c} (%) | | | | | |
|---|---|---|---|---|---|---|---|---|---|---|---|---|
| | | | | | | | Maintaining | | Increased | | Combined | |
| | | Respirable | | | | | Atmospheric | | Aerosol mixing | | Effect | |
| | | Delivered Dose^{b} | | | | | Pressure | | chamber | | (Vented + | |
| | Aerosol | (mg/time increment) | | | | | (Vented) | | (XL) | | XL) | |
| Medicine Cup | mixing | 0-2 | 2-4 | 4-6 | 6-8 | | 0-2 | 6-8 | 0-2 | 6-8 | 0-2 | 6-8 |
| Pressure^{a} | chamber | min | min | min | min | Slope | min | min | min | min | min | min |
| V | L | 5.97 | 6.16 | 6.33 | 6.32 | 0.12 | 3.91^{d} | 19.92^{d} | 11.35^{e} | 12.49^{e} | | |
| NV | L | 5.75 | 5.71 | 5.49 | 5.27 | −0.16 | | | | | | |
| V | XL | 6.65 | 7.07 | 7.01 | 7.11 | 0.13 | 2.27^{f} | 21.23^{f} | 13.15^{g} | 11.28^{g} | 15.71^{h} | 34.90^{h} |

| | | | | | | |
|---|---|---|---|---|---|---|
| NV | XL | 6.50 | 6.44 | 6.37 | 5.87 | −0.20 |

^{a}V: vented (medicine cup reservoir maintained under atmospheric pressure), NV: non-vented (closed system medicine cup reservoir);
^{b}Respirable delivered dose (RDD; mg inhaled pirfenidone in aerosol droplets < 5 microns) during 2 minute simulated inhaled aerosol increments;
^{c}Benefit measured as percent improvement between medicine cup pressure (vented vs. non-vented in either “L” or “XL” configurations), aerosol mixing chamber volume (“L” vs. “XL” in either vented or non-vented configurations), and the combined benefit of a vented “XL” device configuration compared to a non-vented “L” device configuration.
^{d}vented “L” vs. non-vented “L”;
^{e}vented “L” vs. vented “XL”;
^{f}vented “XL” vs. non-vented “XL”;
^{g}non-vented “L” vs. non-vented “XL”;
^{h}vented “XL” vs. non- vented “L”.

The aerosol pirfenidone data presented in Table 4 indicates that the vented XL device configuration delivers a respirable delivered pirfenidone dose of 27.84 mg over 8 minutes (3.48 mg/min; respirable aerosol droplet output rate), with increased rate over the administration period (positive slope) compared to the non-vented L configuration that delivers a respirable delivered pirfenidone dose of 22.22 mg over 8 minutes (2.78 mg/min; respirable aerosol droplet output rate), with increased slowing (negative slope) over the administration period. Moreover, the differential vented XL aerosol mixing chamber 8 benefit increases with time; a benefit of maintaining atmospheric pressure compared to the non-vented configuration. To separate the contribution of each component, the aerosol pirfenidone data presented in Table 4 shows that venting the medicine cup reservoir 3 exhibits only a small benefit in the early stage of pirfenidone nebulized dose administration (about +3.9% in the “L” configuration and about +2.3% in the “XL” configuration). However, the venting benefit increases substantially near the end of administration (to about +19.9% in the “L” configuration and about +21.2% in the “XL” configuration), suggesting that maintaining atmospheric pressure and/or avoiding increased negative pressure that may occur during administration as the dose volume reduces within the sealed, non-vented medicine cup reservoir 3 is strongly beneficial for pirfenidone administration in the drug-device combination described herein.

The data also show that increasing aerosol mixing chamber 8 volume is beneficial. As predicted, this benefit is not dependent upon medicine cup reservoir 3 pressure (about +11 to +13% benefit in all device configurations regardless of medicine cup reservoir 3 configuration). Combining these two device elements demonstrates a substantial additive benefit (from about +16% in the early stage of nebulized dose administration of an aqueous solution of pirfenidone, increasing to about +35%). Thus, including a larger volume aerosol mixing chamber 8 in combination with the vented configuration wherein a nebulizer 1 includes the various options for the vent pathway 4 measurably improves pulmonary deposition.

Moreover, the data of Table 4 demonstrate that the effect of the larger volume aerosol mixing chamber 8 is separate and independent, but synergistic with the design incorporating the vent pathway 4 in the structure of the nebulizer or assembly. Accordingly, the improvement provided by the vented configuration is independent of the additional improvement provided by the enlarged aerosol mixing chamber 8 can be applied to nebulizer designs that have ordinary or smaller aerosol mixing chambers. Furthermore, as the Table 4 data reveals, the ability to avoid a negative slope in the respirable delivered dose rate, as the volume in the medicine cup reservoir 3 is reduced, is separately provided by either or both of the venting structures or the internal volume of the aerosol mixing chamber 8. Also, the various venting structures of the nebulizer 1 are readily applied to different concentrations of pirfenidone described herein, the different medicine cup reservoir 3 fill volumes, a range of respirable delivered dose rates, total respirable delivered doses, daily respirable delivered doses total output rates.

As noted above, one option for the overall assembly for the nebulizer 1 includes an aerosol generator actuation circuit that is not user-controlled, rather is comprised of an activation system where the pressure differential created caused by the intake of a patient breath at the mouthpiece of the nebulizer activates the aerosol generator 7 to convert the aqueous solution of pirfenidone into the therapeutic aerosol. In this configuration, the vented structures also provide the distinct advantage as described herein and shown in the data, even though there is less quantity of the aerosol maintained in the aerosol mixing chamber during administration.

From the data disclosed herein, the preferred device embodiment utilizes either or both of the vented medicine cup reservoir to maintains atmospheric pressure throughout dose nebulization and any size of the greater than L embodiment, or the XL aerosol mixing chamber 8 in combination individually improves the performance across each of the drug-device combinations as described herein.

Clinical study data indicates that this preferred device embodiment V/XL nebulizing 8 mL of a 12.5 mg/mL pirfenidone aqueous solution given twice daily (100 mg device-loaded dose; 200 mg daily dose) is efficacious in slowing-to-stabilizing pulmonary fibrosis progression. Further, nebulizing 4 mL of a 12.5 mg/mL pirfenidone aqueous solution given once a day (50 mg device loaded dose; 50 mg daily dose) is more efficacious than historical placebo, but less efficacious than the 200 mg daily dose. Considering the data in Table 4, the preferred device embodiment having a combination of the vent and larger aerosol mixing chamber (V/XL) provides a total respirable delivered pirfenidone dose of about 27.8 mg in about 8 minutes from 8 mL of a 12.5 mg/mL pirfenidone aqueous solution. By calculation, this delivers about 3.5 mg respirable pirfenidone per minute from a 12.5 mg/mL pirfenidone aqueous solution. Using the non-vented medicine cup reservoir 3 and L aerosol mixing chamber 8 device combination provides a total respirable delivered pirfenidone dose of about 22.2 mg pirfenidone over the same duration and same dosing solution. By calculation, this configuration device delivers about 2.8 mg respirable pirfenidone per minute from a 12.5 mg/mL pirfenidone aqueous solution, or about 25% less per unit time than the preferred V/XL embodiment device. Given pirfenidone activity is concentration dependent, more rapid delivery is required to overcome elimination mechanisms and permit higher pulmonary concentrations and activity.

Because the 50 mg pirfenidone aqueous solution dose delivered using the preferred embodiment device loaded was efficacious, albeit less than the 200 mg daily device loaded dose, it is considered that a lower dose may also contain efficacious content. Given the data described herein, it is predicted that a fifty percent lower daily dose (25 mg) would be non-efficacious. By calculation and using the preferred V/XL embodiment device, a 25 mg device-loaded dose of a 12.5 mg/mL pirfenidone aqueous solution would provide a respirable delivered dose of about 7 mg at a similar 3.5 mg per minute respirable delivered dose rate as the 100 mg BID (200 mg daily) device loaded dose. Taken together, using the preferred V/XL device embodiment, a daily dose level greater than 25 mg, wherein the pirfenidone respirable delivered dose is greater than about 7 mg and delivered at a rate of more than 2.8 mg per minute.

The drug device combination above theoretically delivers as much as 12.5 mg/mL quantity of pirfenidone, although assuming a fifty percent respirable delivered dose, the total delivery would be a rate of 6.25 mg per minute. Clinical data demonstrates that using the drug device combination above delivers approximately 5.625 mg per minute, although those numbers vary considerably based on external factors. Accordingly, the improvement in the therapeutic administration using the drug-device combination of the invention can be described as the added treatment value of administering aerosol pirfenidone at a rate between 2.8 mg per minute and 6.25 mg per minute with values approximating 5.625 mg per minute confirmed by clinical trial.

### Example 2. Human Pharmacokinetic Modeling

Employing the Example 1 data, a human pharmacokinetic model was run to compare the minimum effect of medicine cup reservoir pressure and aerosol mixing chamber volume on predicted rate for increased pirfenidone lung tissue and lung epithelial lining fluid (ELF) concentrations (mcg/mL pirfenidone per minute inhaled aerosol administration) and exposure (mg·hr/L pirfenidone per minute inhaled aerosol administration). The results are shown in Table 5.

TABLE 5
Modeled pirfenidone delivery and accumulation rate
in human lung tissue and epithelial lining fluid

| | | | | | | ELF Delivery Benefit^{c} (%) | | | | | |
|---|---|---|---|---|---|---|---|---|---|---|---|
| | | | | | | | | Increased | | | |
| | | | | | | Maintaining | | Aerosol | | Combined | |
| | | Pirfenidone Respirable | | | | Atmospheric | | mixing | | Effect | |
| | | Delivery Rate^{b} | | | | Pressure | | chamber | | (Vented + | |
| | Aerosol | Lung Tissue^{d} | | ELF^{e} | | (Vented) | | (XL) | | XL) | |
| Medicine Cup | mixing | mcg/ | mg · hr/ | mcg/ | mg · hr/ | mcg/ | mg · hr/ | mcg/ | mg · hr/ | mcg/ | mg · hr/ |
| Pressure^{a} | chamber | mL/min | L/min | mL/min | L/min | mL/min | L/min | mL/min | L/min | mL/min | L/min |
| V | L | 3.71 | 0.06 | 9.80 | 0.16 | 36.56^{f} | 34.36^{f} | 9.39^{g} | 10.60^{g} | | |
| NV | L | 2.72 | 0.04 | 7.18 | 0.12 | | | | | | |
| V | XL | 4.07 | 0.06 | 10.72 | 0.18 | 30.24^{h} | 29.27^{h} | 14.69ⁱ | 14.96ⁱ | 49.37^{j} | 48.60^{j} |
| NV | XL | 3.11 | 0.05 | 8.23 | 0.14 | | | | | | |

^{a}V: vented (medicine cup reservoir maintained under atmospheric pressure), NV: non-vented (closed system medicine up reservoir);
^{b}Pirfenidone respirable delivery rate (mcg/mL or mg · hr/L pirfenidone added to either ELF or lung tissue per minute nebulized AP01 inhaled aerosol administration);
^{c}ELF benefit measured as percent increased pirfenidone mcg/mL or mg · hr/L added to either ELF or lung tissue per minute nebulized pirfenidone inhaled aerosol administration between medicine cup pressure (vented vs. non-vented in either “L” or “XL” configurations), aerosol mixing chamber volume (“L” vs. “XL” in either vented or non-vented configurations), and the combined benefit of a vented “XL” device configuration compared to a non-vented “L” device configuration;
dLung tissue: modeled pirfenidone deposition into 600 g human lung tissue;
^{e}ELF: modeled pirfenidone deposition into 20 mL human epithelial lung fluid;
^{f}vented “L” vs. non-vented “L”;
^{g}vented “L” vs. vented “XL”;
^{h}vented “XL” vs. non-vented ″XL”;
ⁱnon-vented “L” vs. non-vented “XL”;
^{j}vented “XL” vs. non-vented “L”.

The modeled pirfenidone pharmacokinetic data presented in Table 5 shows that establishing the vent pathway 4 in the medicine cup reservoir 3 exhibits a strong increase in lung tissue and ELF pirfenidone deposition per unit time (about +34% to +36% in the “L” configuration and about +29% to +30% in the “XL” configuration), demonstrating that maintaining atmospheric pressure in the medicine cup reservoir 3 during nebulization and inhaled aerosol administration substantially increases pirfenidone lung deposition per unit time. The data further indicates that increasing aerosol mixing chamber 8 volume also strongly increases pirfenidone lung deposition per unit time (about +9% to +10% in the “L” configuration and about +14% to +15% in the “XL” configuration), demonstrating that increasing the aerosol mixing chamber 8 volume also substantially increases pirfenidone lung deposition per unit time. Combining these two device elements demonstrates a substantial additive benefit, wherein ELF pirfenidone concentration rate (mcg/mL/min) and exposure rate (mg·hr/L/min) increase +49% over the device configuration lacking these features (non-vented “L” configuration). Taken together, maintaining atmospheric pressure in the medicine cup reservoir 3 throughout nebulization and inhaled administration or increasing aerosol mixing chamber 8 volume alone or when combined together substantially increases lung-delivered ELF or lung tissue pirfenidone Cmax or AUC, key pharmacokinetic properties important for therapeutic effect.

### Example 3. Nebulizer Specifications and Human Administration

To establish an aerosol generator (head) specification meeting the desired delivery of 8 mL aqueous pirfenidone solution in the assembled nebulizer device 1 not exceeding 16 minute delivery time (or an 0.5 mL/min output rate), a correlation study between the head-only performance with 0.9% NaCl (saline) and the assembled nebulizer device 1 (vented, XL configuration) performance with aqueous pirfenidone was conducted. Using heads only, the aerosol characteristics total output rate (TOR), volumetric median diameter (VMD) and geometric standard deviation (GSD) were performed under constant negative pressure (−250 mbar; relative to atmospheric pressure) and under ambient, atmospheric pressure conditions (0 mbar; relative to atmospheric pressure). These results were then compared to the same performance values (plus addition of nebulization time) of saline and aqueous pirfenidone solution in the assembled nebulizer device 1.

Using Sympatec Helos instrumentation for measuring aerosol droplet size distribution, aerosols were generated from 53 aerosol heads exhibiting a pre-screened VMD less than 5 microns. These heads were either tested alone using a special apparatus (saline −250 mbar or 0 mbar) or in assembled devices (saline and aqueous pirfenidone; 8 mL vented medicine cup reservoir 3 and XL aerosol mixing chamber 8 configuration).

The results of TOR testing indicate head-only saline testing at −250 mbar and 0 mbar with saline and device testing with aqueous pirfenidone are similar. The average TOR value of device testing with saline is slightly decreased in comparison. The lowest standard deviation between the 53 aerosol heads occurred during device testing with aqueous pirfenidone. Results are shown in Table 6.

TABLE 6
Average Total Output Rate (TOR)

| | |
|---|---|
| Head-Only (g/min) | |
| Saline | Vented, XL Device (g/min) |

| | −250 mbar | 0 mbar | Saline | Pirfenidone |
|---|---|---|---|---|
| Min | 0.345 | 0.282 | 0.191 | 0.382 |
| Max | 1.105 | 1.340 | 1.098 | 1.049 |
| Average | 0.759 | 0.793 | 0.636 | 0.782 |
| SD | 0.249 | 0.350 | 0.294 | 0.178 |

Based upon gravimetric assessment, the data in Table 6 predicts that the vented, XL device configuration will have a TOR at 0 mbar of 0.382 g/min (weight of pirfenidone aqueous solution per unit time; equivalent to approximately 0.38 mL/min). The correlation between saline head-only TOR measured at 0 mbar and saline vented XL device TOR provides an RSQ value of 0.9468 and significantly improved correlation compared to head-only at −250 mbar. This data further predicts a 95% confidence level for the correlation between saline head-only TOR measured at 0 mbar and saline vented XL device TOR. To define the lower specification limit for head-only saline TOR at 0 mbar predicting a minimum TOR of 0.35 g/min in vented XL device, the corresponding saline head-only TOR was 0.58 g/min, rounded up to 0.6 g/min.

The correlation between saline head-only TOR measured at −250 mbar and saline vented XL device TOR provides an RSQ value of 0.7556 and similar TOR values measured at −250 mbar (e.g. 0.9 g/min) resulted in varying TOR values measured in the device between 0.65-1.0 g/min. This data further predicts a 95% confidence lower specification limit for head-only saline TOR at −250 mbar 0.74 g/min.

The correlation between aqueous pirfenidone vented XL device TOR and saline vented XL device TOR provided an RSQ value of 0.7587. Based upon duration to nebulize a set volume, the results of dosing time for nebulizing 8 mL saline and 8 mL aqueous pirfenidone solution are presented in

Table 7. The average nebulization time for 8 mL aqueous pirfenidone was 3 minutes faster than for 8 mL saline, providing a minimal delivery time (fastest output rate) of 6.35 min to nebulize 8 mL aqueous pirfenidone solution, or about 1.26 mL/min, and a maximum delivery time (minimum output rate) of 14.58 min, or about 0.55 mL/min. These data supported the device specification of an output rate of at least 0.5 mL/min. The standard deviation between the 53 tested aerosol heads was lower when nebulizing an aqueous solution of pirfenidone. The correlation between aqueous pirfenidone nebulization time and saline nebulization time provides an RSQ value of 0.7125. At a 95% confidence level, the correlation between the saline vented XL device TOR>0.350 g/min will result in a nebulization time for 8 mL aqueous pirfenidone in the same vented, XL device configuration of less than 14.6 minutes.

TABLE 7
Average Nebulization Time

Vented, XL Device (min)

| | Saline | Aqueous pirfenidone |
|---|---|---|
| Min | 6.33 | 6.35 |
| Max | 22.00 | 14.58 |
| Average | 11.78 | 8.78 |
| SD | 4.84 | 1.90 |

The reduction in the average nebulization time and the reduction in the standard deviation of average nebulization times provides an important therapeutic advantage because the delivery of more medication in less time provides a therapeutic advantage. Furthermore, the reduction in the standard deviation in the delivery time means that the delivery time from patient to patient is likely to the far more reliable such that differences in nebulizer performance from device to device is reduced resulting in more reliable patient care.

VMD results are shown in Table 8. Results show that establishing a vent pathway 4 in the nebulizer device 1 increases the aerosol droplet population median size. As a mass median diameter, these results are the average number of aerosol droplets generated from this device configuration. From Example 1, although venting is shown to increase the aerosol population size, the respirable dose remains the same.

TABLE 8
Average Volumetric Median Diameter (VMD)

| | Head-Only (μm) | | | |
|---|---|---|---|---|
| | Saline | | Vented, XL Device (μm) | |
| | −250 mbar | 0 mbar | Saline | aqueous pirfenidone |
| Min | 3.19 | 3.26 | 3.41 | 3.45 |
| Max | 3.91 | 4.34 | 4.38 | 4.60 |
| Average | 3.52 | 3.89 | 3.90 | 4.05 |
| SD | 0.14 | 0.19 | 0.21 | 0.25 |

The correlation between head-only saline VMD at 0 mbar and saline vented XL device VMD provides an RSQ value of 0.5634. To define the lower and upper specification limits for head-only saline VMD at 0 mbar predicting the specified device VMD with saline of 3.6-4.8 μm, the intersection points at 3.6 μm and 4.8 μm corresponded with a head-only VMD values at 0 mbar of 3.86-4.60 μm (3.9-4.6 μm).

The correlation between saline VMD measured head-only at −250 mbar and saline vented XL device VMD provides an RSQ value of 0.377. The correlation between AP01 vented XL device VMD and saline vented XL device VMD provides an RSQ value of 0.4885. GSD results are shown in Table 9.

TABLE 9
Average Geometric Standard Deviation (GSD)

| | Head-Only (μm) | | | |
|---|---|---|---|---|
| | Saline | | Vented, XL Device (μm) | |
| | −250 mbar | 0 mbar | Saline | Aqueous Pirfenidone |
| Min | 1.44 | 1.50 | 1.54 | 1.54 |
| Max | 1.87 | 1.64 | 1.74 | 1.72 |
| Average | 1.48 | 1.57 | 1.63 | 1.62 |
| SD | 0.06 | 0.03 | 0.05 | 0.04 |

A basis of data was generated and the necessary steps were carried out to fulfill the Design Input Requirement (DIR) of ensuring a nebulization time of less than or equal to 16 minutes when nebulizing 8 mL aqueous pirfenidone with the vented XL device. A head-only TOR of 0.740 g/min measured at −250 mbar and of 600 mg/min measured at 0 mbar conditions with saline were identified to ensure a saline vented XL device TOR of 0.350 g/min. A head-only VMD of 3.9-4.6 μm measured at 0 mbar with saline correlates with the specified device VMD with saline of 3.6-4.8 μm. An improved correlation between the head-only and device aerosol performance was achieved with the quality control measurements at 0 mbar. All aerosol heads fulfilling the defined criteria resulted in a nebulization time of below 16 minutes when nebulizing 8 mL AP01 in the vented XL device.

From this data, a new head class was established with quality control testing at 0 mbar and an aerosol saline head-only specification TOR>0.600 g/min and VMDVMD=3.9-4.6 μm.

In clinical studies, inhaled aqueous pirfenidone was administered to 91 IPF patients daily for 6 months. In this study, patients used the 8 mL, vented medicine reservoir cup 3, XL aerosol mixing chamber 8 configuration nebulizer 1 to receive either a 50 mg (4 mL aqueous pirfenidone) dose once-daily or a 100 mg (8 mL aqueous pirfenidone) dose twice-daily. Day 1 mean duration for study drug administration was 4.9 min for the 50 mg dose and 8.8 min for the 100 mg dose.

### Example 4. Nebulizer Design and Specifications for Aerosol Mixing Chambers and Venting to Ambient Pressure

The following descriptions of structures, functions, and mechanical expedients for accomplishing the advantage of the present invention are not to the exclusion of substitutions achieving equivalent designs having the same mechanical and functional capabilities of the present invention.

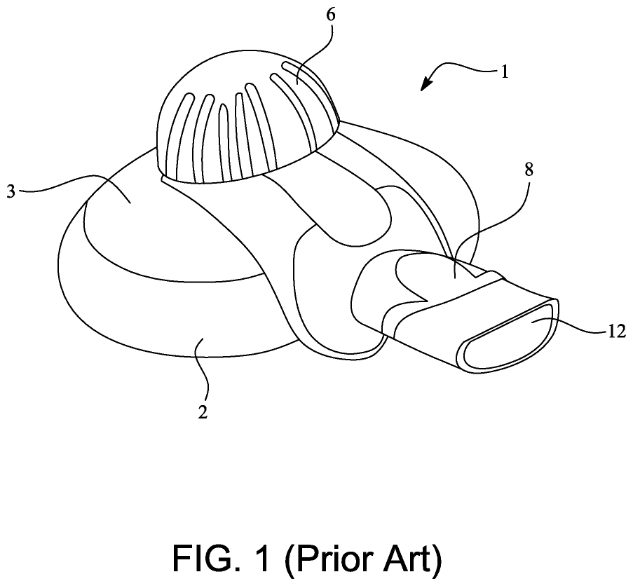

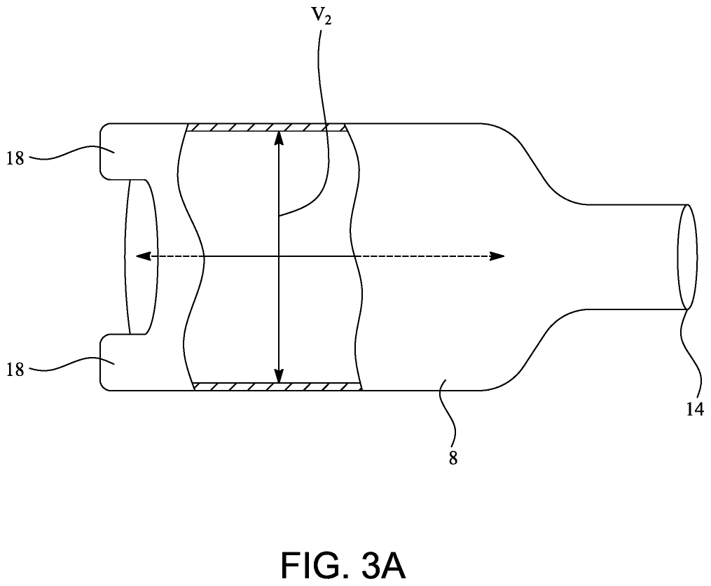

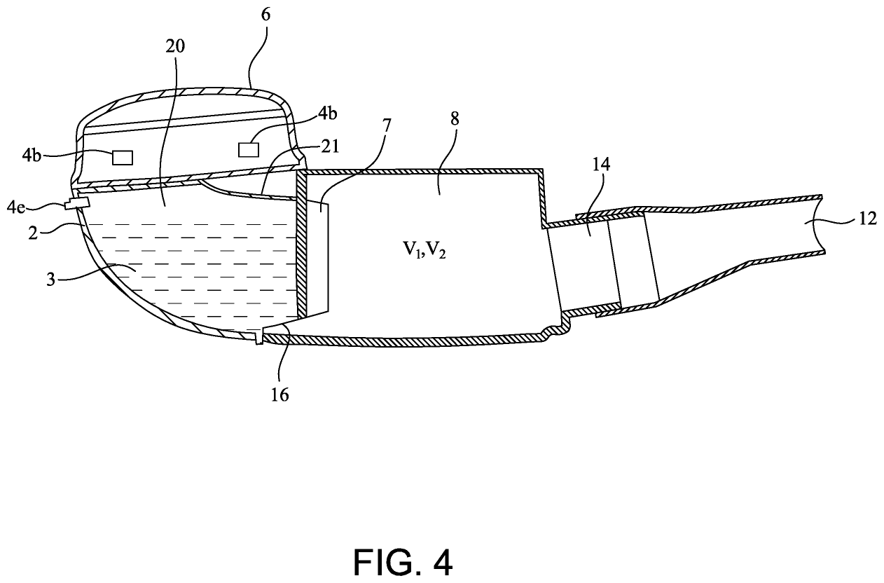

FIG. 1 is a conventional prior art nebulizer 1 having a housing 2, an internal medicine cup reservoir 3, a cap 6 for sealing of the reservoir 3, a conventionally sized “L” aerosol mixing chamber 8 and a mouthpiece 12 for inspiration of the active pharmaceutical ingredient (API). The aerosol generator (not shown) is disposed inside the housing 2 of the nebulizer 1 between the medicine cup reservoir 3 and the aerosol mixing chamber 8. Activation of the aerosol generator 7 by a patient creates an aerosol of the aqueous API solution disposed in the reservoir 3 that accumulates in the aerosol mixing chamber 8 until the aerosol is inhaled by the patient through the mouthpiece 12. As described in more detail in connection with FIG. 4 below, during the ordinary operation of a prior art nebulizer 1, the process of converting the liquid contained in the medicine cup reservoir 3, combined with an orientation wherein the medicine cap 6 seals the housing 2 about the opening of the medicine cup reservoir 3, creates a negative pressure in the headspace 20 of the medicine cup reservoir 3 internal of the housing 2.

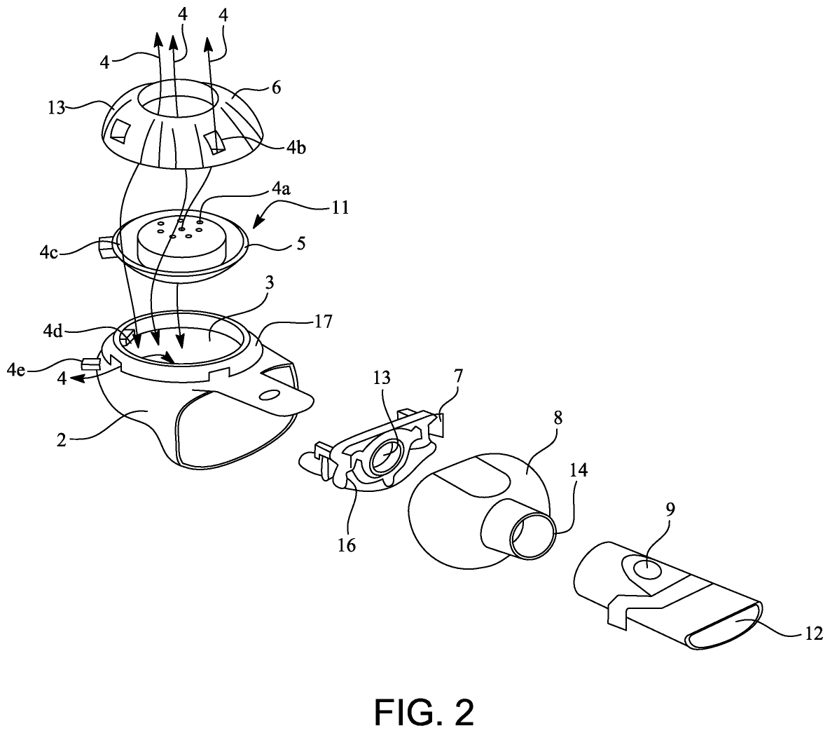

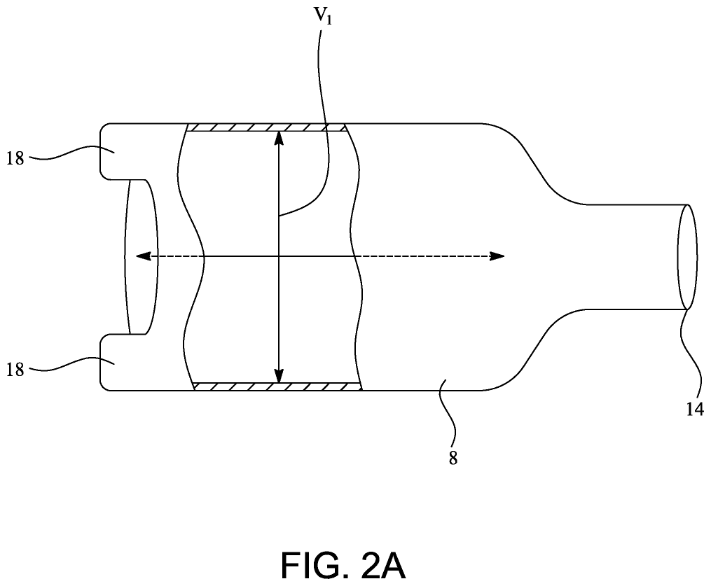

FIG. 2 is an exploded view of the nebulizer 1 of the present invention and is comprised of several discrete structural elements that also have subunits for some assemblies as described below. The body of the nebulizer 1 has a housing 2 that contains the aerosol generator 7 that is disposed between the medicine cup reservoir 3 and the aerosol mixing chamber 8. The aerosol generator 7 is mounted between the housing 2 of the nebulizer 1 and the aerosol mixing chamber 8. Although the configurations may vary, the aerosol generator 7 may have a mating fixture 16 designed to seal the aerosol generator against the corresponding structure on the aerosol mixing chamber 8. The aerosol generator 7 may have a centrally disposed vibrating mesh membrane 13 that generates an aerosol from aqueous formulations of the API placed in the medicine cup reservoir 3 using a liquid pathway that places the aqueous formulation in fluid communication with the aerosol generator 7. The housing 2 also contains at least a portion of the medicine cup reservoir 3 and together with the medicine cap 6 contains the aqueous solution and encompasses air headspace 20 (see FIG. 4) above the aqueous solution. The medication reservoir cap 6 typically has an engaging mechanism, such as a threaded or rotational closure that engages with the opening allowing access to the medicine cup reservoir 3 disposed within the housing 2 and that functionally closes the opening to form a fluid seal containing the medicine cup reservoir 3 once the aqueous solution of the API is disposed in the reservoir 3, but, as described below, incorporates a vent pathway 4 that maintains ambient pressure between the external atmosphere and the headspace 20 contained above the aqueous solution contained within the medicine cup reservoir 3.

As described above and in detail below, internal structures of the housing 2 are configured such that the aqueous solution contained in the medicine cup reservoir 3 has a fluid pathway (not shown) between the medicine cup reservoir 3 and the aerosol generator 7 prior to activation of the aerosol generator 7 by the patient. The medicine cap 6 may have a variety of different structural alternatives that achieve the function of containing the aqueous solution of the API in the medicine cup reservoir 3 and may comprise a portion of the vent pathway 4. Most typically, simple gravity fed fluid pathway funnels the aqueous solution of the API to bring the solution in contact with the aerosol generator 7, and particularly the vibrating mesh membrane 13. Once operation of the nebulizer 1 is activated by the patient, the aerosol generator 7 continues producing a fine particle fraction of the aerosol until all of the aqueous solution contained in the medicine cup reservoir 3 is consumed or until a predetermined time period is reached based on the volume and concentration of the aqueous solution as prescribed to an individual patient and consistent with the aerosol delivery parameters for fill volume, total dosage, respirable dose delivery rate, and other parameters as described herein. Accordingly, each specific formulation and delivery parameter described in the foregoing Tables and accompanying text is readily applied to the improved nebulizer designs described in these Figures.

In another embodiment, the operation of the aerosol generator 7 may be triggered by a breath-actuated circuit that senses the changing pressure from the inhalation function by the patient and produces a fine particle fraction of the API in response to activation of the breath-actuated circuit.

As described in FIGS. 2-5 several embodiments of a vent pathway 4 are disclosed to maintain ambient pressure in the medicine cup reservoir 3 as the aqueous solution is converted to aerosol. The term “vent pathway” describes the combination of structures that permit ambient pressure to be maintained in the headspace 20 above the aqueous solution disposed in the medicine cup reservoir 3. These structures may include openings, ports, or apertures (for example elements 4a, 4b, 4c, 4d, and 4e) that include both the open structure and surrounding structural features of any of the housing, sealing element, that provide the opening and the length of the vent pathway 4 to ambient pressure. It is also possible for the vent pathway to traverse the aerosol generator 7 in the manner shown in U.S. Pat. No. 8,387,895. In the embodiment of FIG. 2, the medicine cap 6 is paired with a closure 11 disposed between the housing 2 of the nebulizer 1 and the medicine cap 6 to provide a fluid seal superior to the medicine cup reservoir 3 and partially defining the headspace 20. The closure 11 has an annular flange 5 to circumferentially engage the corresponding annular configuration of the upper portion 17 of the housing 2 and the annular bottom edge of the medicine cap 6 to form a fluid seal thereabout. In this embodiment, the vent pathway 4 is created by the combination of the passages 4a that traverse a top surface of the closure 11 and the ports 4b that traverse the outer circumferential edge 13 of the medicine cap 6 to permit access of ambient air into the space between the annular flange 5 of closure 11 and the port 4b of the medicine cap 6. In this configuration the top of the medicine cap 6 may be solid as opposed to opened as shown in FIG. 2. The open configuration for the medicine 6 is preferably combined with an alternate structure that prevents spillage of the solution out of the medicine cup reservoir 3 such that the vent pathway is defined as “occluded” as defined below.

If the medicine cap 6 is open to permit inflow of ambient air, then the structures that comprise vent pathway 4 would be offset from the opening in the top of medicine cap 6 to avoid liquid solution for exiting the medicine cup reservoir 3, for example by including the closure 11 having the notch 4c in the annular edge 5 rather than the ports 4a in the upper portion thereof. As noted below, the vent pathway 4 is preferably occluded to allow air to flow but to prevent and potential spillage of liquid through the vent pathway. The occlusion may be provided by the orientation of any of the housing 2, the closure, the orientation and structure of the medicine 6 or any combination of the above. Separately, the occluded vent pathway 4 may be established by a structural member (not shown) disposed within an opening of the vent pathway 4 itself either internal to one of the openings or along a portion of the path of the vent pathway 4 such that ambient pressure airflow is maintained while preventing the passage of fluid. Thus, in this embodiment, the vent pathway 4 is comprised of port 4b, and passages 4a such that external ambient air can flow therethrough and into the medicine cup reservoir 3 as the aqueous solution of the API is nebulized and the volume maintained within the reservoir 3 is reduced. In this configuration, the pressure in the medicine cup reservoir remains at or near ambient levels and the vent pathway 4 prevents the development of negative pressure in the medicine cup reservoir 3.

In the embodiment of FIG. 2, the vent pathway 4 is sometimes described herein as “occluded” because no linear pathway exists from the medicine cup reservoir 3 to the ambient environment external of the nebulizer 1 to avoid any possibility for the aqueous solution in the medicine cup reservoir 3 from spilling out the nebulizer device through the vent pathway 4. In this embodiment, the passages 4a and the ports 4b are offset, meaning not in a linear alignment, such liquid that may pass through the passages 4a cannot also pass through the ports 4b. Accordingly, the combination of the individual elements that make up the vent pathway 4 are preferably arranged so that no linear alignment can exist between the medicine cup reservoir and ambient air among the components of the vent pathway 4 when the nebulizer device 1 is assembled. Additional configurations for the vent pathway 4 are described in the accompanying FIG. 4.

Referring again to FIG. 2 and specifically in connection with FIG. 2A, the invention includes an aerosol mixing chamber 8 having a large internal volume to increase the performance of the nebulizer by increasing the delivery rate of a population of respirable aerosol droplets during aerosolization of the aqueous solution of the API. As noted above, increasing the volume of the aerosol mixing chamber 8 reduces aerosol inter-droplet collisions of the freshly generated API aerosol limits impaction of the aerosol population with the wall of the aerosol mixing chamber 8, and limits droplet growth and/or rainout during the exhalation phase, prior to inhalation, or during inhalation. The larger volume of the aerosol mixing chamber 8 also enables more aerosol to accumulate during the exhalation phase. Referring to FIG. 2A, the aerosol mixing chamber 8 has an internal volume V1 defined by the length and diameter of the aerosol mixing chamber 8 and is designated 1′ and is generally greater than 49 mL, although in combination with the vented nebulizer 1 or in combination with the breath-actuated nebulizer system, therapeutic advantage may still be achieved with a aerosol mixing chamber 8 lower than 49 cm³. Accordingly, the specific embodiment of both FIGS. 2 and 2A can be combined with the vent pathway 4 structures shown in FIG. 2 without regard to the particular volume of the aerosol mixing chamber 8.

The larger volume L aerosol mixing chamber 8 having internal volume V1 is joined to the nebulizer housing 2 at mating fixture 16 and may have connector 14 to engage the mouthpiece 12. The internal volume of the aerosol mixing chamber 8 L is defined as the volume available for containing a respirable delivered dose of an aerosol created by the aerosol generator 7 and maintained between the aerosol generator 7 and the mouthpiece 12 within the aerosol mixing chamber 8 until inhaled.

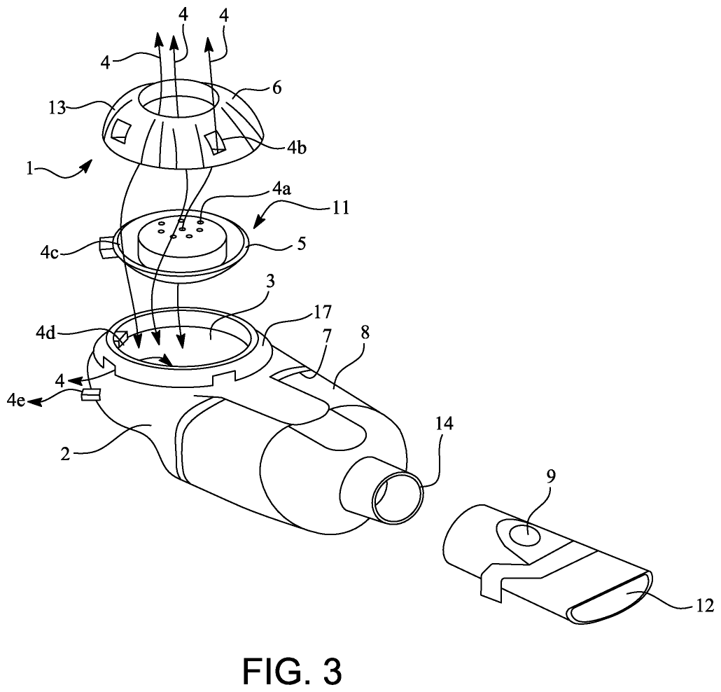

Alternate testing for the V1 dimension demonstrates that increasing internal volumes for the aerosol mixing chamber 8 provides advantages with internal volumes greater than 49 ml, greater than 60 mL, greater than 70 mL, greater than 80 mL, greater than 90 mL, greater than 100 mL, greater than 110 mL, greater than 120 mL, greater than 130 mL, greater than 140 mL, and at least as high as an internal volume V2 of 150 mL and are designated XL at volumes greater than 98 cm³ (see FIG. 3A). As disclosed by the data provided in the accompanying Tables and text above, a synergistic relationship exists between the creation of the ambient air vent pathway 4 to the medicine cup reservoir 3 and the increased size of the aerosol mixing chamber 8 such that individual embodiments of the vent pathway 4 may be readily applied to any of the particular configurations or sizes of the aerosol mixing chamber 8 as well as the various aqueous pirfenidone concentrations, filled dose volumes, respirable delivered doses, respirable delivered dose rates, total daily doses, amounts per individual dose, and varying total output rates for the nebulizer 1.

Referring to FIGS. 3 and 3A, the large volume XL aerosol mixing chamber 8 having internal volume V2 between 98 cm³ and above is joined to the nebulizer housing 2. The internal volume V2 of the aerosol mixing chamber 8 is defined as the volume available for containing a respirable delivered dose of an aerosol created by the aerosol generator 7 and maintained in the aerosol mixing chamber 8 between the aerosol generator 7 and the mouthpiece 12 within the aerosol mixing chamber 8 until inhaled.). As disclosed by the data provided in the accompanying Tables and text above, a synergistic relationship exists between the creation of the ambient air vent pathway 4 to the medicine cup reservoir 3 and the increased size of the aerosol mixing chamber 8 such that individual embodiments of the vent pathway 4 may be readily applied to any of the particular configurations or sizes of the aerosol mixing chamber 8 as well as the various aqueous pirfenidone concentrations, filled dose volumes, respirable delivered doses, respirable delivered dose rates, total daily doses, amounts per individual dose, and varying total output rates for the nebulizer 1.

FIG. 4 is a cross-section of the drug-device combination of the present invention. In the operative orientation and assembly, the medicine cap 6 is fixedly and removably attached to the upper portion of the housing 2 following filling of the medicine cap reservoir 3 with the aqueous solution. Ports 4B, disposed in the medicine cap are disposed in the side wall of the medicine cap 6 and provide a vent pathway 4 between the headspace 20 of the medicine cup reservoir 3 and ambient air. An alternate vent pathway 4 is comprised of a port 4e disposed in the side wall of the nebulizer housing 2 and similarly providing a vent space between the headspace 20 and ambient air. All of the vent pathway configurations disclosed in FIGS. 2 and 3 above are also applicable to the nebulizer design of FIG. 4. The port 4e is disposed above the fluid level of the aqueous solution contained in the medicine cup reservoir 3 and may be occluded along its internal length to avoid spillage of the contents of the reservoir 3.

In the assembled state, a receiving portion 16 of the housing 2 engages either of the aerosol generator 7 or at mating fixture 18 of the aerosol chamber 8 or both to fix the position of the members of the assembly and to contain the aerosol generator 7. Either of the aerosol chamber 8 or the housing of the nebulizer 2 may engage either or both sides of the aerosol generator 7 about the periphery. The main constraint on the engagement features of the housing 2, aerosol generator 7, an aerosol mixing chamber 8, is to avoid obstructing any portion of the fluid delivery pathway between the medicine cup reservoir 3 and the operative portion of the aerosol generator 7, specifically the vibrating mesh membrane 13. Once the aqueous solution in the medicine cup reservoir 3 is converted into the respirable delivered dose of the API and is maintained inside the inner volume V1-V2 of the aerosol mixing chamber 8 of embodiments L and XL having an expanded internal volume, the respirable delivered dose of the API is then inhaled by the patient. Typically, the patient performs a step of triggering a circuit that activates the aerosol generator 7 which operates as long as there is fluid in the medicine cup reservoir 3, or as a function of programming embedded in the circuitry of the nebulizer 1 that operates according to the parameters of the aqueous solution, such as fill volume, concentration, and dosage or dosage rat. As noted above, in some embodiments the triggering of the aerosol generator 7 may be tied to a signal that is breath actuated by the intake of a breath by the patient to trigger the activation of the aerosol generator 7—in such a configuration, the added volume of the aerosol chamber 8 by the L/XL embodiments may be optional.

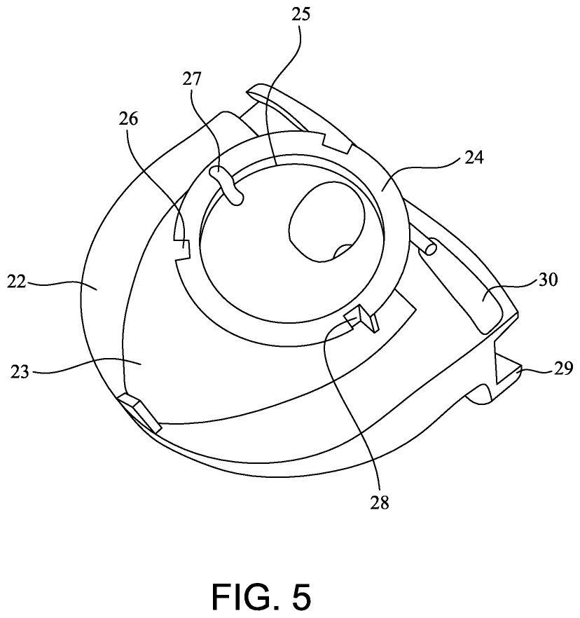

FIG. 5 is an integrated nebulizer assembly comprising a vented container 24 holding the aqueous solution of the API and shaped and designed to be placed within the medicine cup reservoir 3 and to engage the nebulizer 1 to establish the vent pathway 4 in similar fashion as above but with the vent pathway 4 traversing a portion of the vented container 24 rather than being incorporated in the structure of the nebulizer 1 itself. In this fashion, a non-vented nebulizer can be converted into a vented nebulizer 1 assembly. Aqueous API maintained in the vented container 24 has the same fluid pathway to the aerosol generator 7 as the other embodiments described herein. The vented container may have a portion that is susceptible to puncture or a sealing enclosure that can be manually opened to cause the aqueous solution to enter the medicine cup reservoir. Similarly, placement of the vented container 24, attachment to the housing 22, or affixation of a conformingly shaped medication cap can create an opening in the vented container 24 to cause the liquid to enter the medication cup reservoir 3. The vented container 24 is preferably designed and shaped to sealingly engage the body of the housing 22, and preferably in combination with the medicine cap 6. The housing 22 may have a special receptacle 23 shaped to accommodate the outer dimensions of the vented container 24 about any or all of the periphery thereof. The shape of the housing 22 may have a receiving structure (not shown) that engages the outer portion of the vented container to seal the medicine cup reservoir 3 against spillage of the aqueous API solution from the vented container 24. In similar fashion, the vented container 24 may have an outer edge 25 that engages an annular opening in the housing 22 such as screw threads or other mechanical expedient to permit fixed attachment of the vented container 24 to the housing 22 or to an upper portion of medication cup reservoir 3 to allow fluid connection between the vented container 24 and the medicine cup reservoir 3. The vented container 24 may also have a fixed ability to rotate around the opening of the housing 22 such that the rotational orientation of the vented container 24 is fixed relative to the housing 22 or the medicine cup reservoir 3, for example to engage or provide a portion of a vent pathway 4 that is positioned in either or both of the vented container 24 or a portion of the housing 22. The rotation may be fixed by a stop or detent 28 disposed in an upper edge of the vented container 24.

The vent pathway 4 may be provided entirely by an opening or orifice 27 placed in the body of the vented container 24 or may be part of an integrated vent pathway 4 comprised of an opening, such as the notch 27, and a mating portion of the housing 22. For example, a vent internal to the vented container (not shown) may establish a vent pathway 4 from the anterior of the vented container 24 to an external fixture 30 disposed in the housing 22 that provides a vent pathway 4 to ambient pressure. Similarly, a vent opening 27 may be placed in any portion of the vented container 24, such as an upper circumferential edge of the housing 22 or may pass laterally to establish a vent pathway 4 allowing ambient pressure in the headspace 20 through a groove or channel 30 formed in an upper portion of the housing 22 or through the body of the housing 22 to a dedicated vent opening 29 proximate to the portion of the housing 22 that engages the aerosol generator 7.

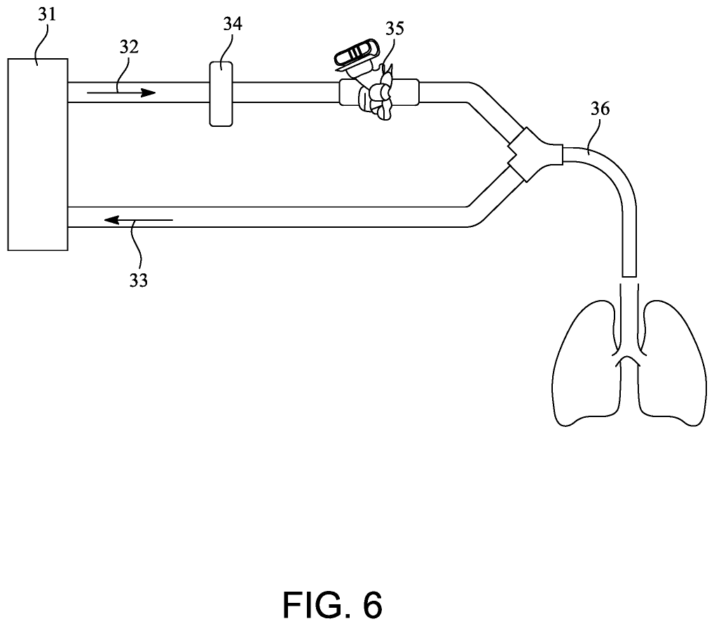

FIG. 6 is a schematic of a system of the present invention comprised of a complete airway including a ventilator 31, inspiratory 32 and expiratory limbs 33, a humidifier 34, an in-line vented nebulizer s5 and fixture 66 for operably connecting the system to a patient.

The ventilator system typically has an airway that extends from the pressure generating components of the ventilator through the airway and into the wye fixture that terminates at the patient. The in-line nebulizer may be placed at any point in the airway between the positive pressure generating mechanics and the patient, however the placement of the nebulizer proximate to the patient near the ventilator wye piece is preferred. In practice, a patient is connected to a ventilator for breathing assistance and the ventilator system is adjusted to provide for a continuous and controlled airflow based on known physiological parameters. The API formulation described above is introduced into the medicine cup reservoir 35 in the in-line nebulizer and is stored therein until delivery. To administer the aerosol, the in-line nebulizer is connected to the airway of the ventilator and the aerosol generator 37 is activated to create the aerosol mist. Upon activation, as with the nebulizer embodiments 1 above, the in-line nebulizer may have a vibrating mesh or membrane 16 that has numerous apertures formed therein to produce particles of a defined size from the API solution.

The position of the in-line vented nebulizer 35 is most proximate to the patient and as close as the configuration of the ventilator will permit. The humidifier 34 and the vented in-line nebulizer 35 are both joined to the airway circuit of the ventilator 31 by a fixture 36 that is sealed at each point of attachment to the inspiratory limb 2 such that additional air is not introduced into the inspiratory limb 32 during inspiration by the patient. The API is introduced into the vented in-line nebulizer 35 for administration to the patient. The humidifier 34 and/or the nebulizer 35 may be activated by program, by patient inspiration or may be continuous during administration of the API aerosol.

The in-line vented nebulizer 35 is designed to remain in the ventilator circuit for the entire treatment course. The in-line vented nebulizer 35 would be inserted near the distal end of the inspiratory tubing to work with any positive pressure ventilator. Unlike a jet aerosol device, it would not introduce any additional air to avoid hyperinflation or barotraumas in a patient. Preferably, the nebulizer 35 is sealed in the airway except for the vent pathway 4 to prevent additional airflow from being introduced. In this configuration, movement of air through the pathway of the ventilator combines humidified air and the aerosol containing the API and may be triggered by patient inspiration or as part of a continuous or programmed delivery protocol such that the nebulizer is in intermittent or continuous operation during administration of the API formulation.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible.

Those of ordinary skill in the art will readily recognize, in light of this description, the many variations of suitable dip casting procedures, pressures, and temperatures that are not stated here yet are suitable to fabricate the prosthetic heart valves described herein. Likewise, those of ordinary skill in the art will also recognize, in light of this description, the alternatives to dip casting that can be used to fabricate the prosthetic heart valves described herein.

As used herein and in the appended claims, the singular forms “a”, “an”, and “the” include plural referents unless the context clearly dictates otherwise.

Where a range of values is provided, each intervening value, to the tenth of the unit of the lower limit unless the context clearly dictates otherwise, between the upper and lower limit of that range and any other stated or intervening value in that stated range, is encompassed within the disclosure and can be claimed as a sole value or as a smaller range. Where the stated range includes one or both of the limits, ranges excluding either or both of those included limits are also included in the disclosure.

Where a discrete value or range of values is provided, that value or range of values may be claimed more broadly than as a discrete number or range of numbers, unless indicated otherwise. For example, each value or range of values provided herein may be claimed as an approximation and this paragraph serves as antecedent basis and written support for the introduction of claims, at any time, that recite each such value or range of values as “approximately” that value, “approximately” that range of values, “about” that value, and/or “about” that range of values. Conversely, if a value or range of values is stated as an approximation or generalization, e.g., approximately X or about X, then that value or range of values can be claimed discretely without using such a broadening term.

However, in no way should this specification be interpreted as implying that the subject matter disclosed herein is limited to a particular value or range of values absent explicit recitation of that value or range of values in the claims. Values and ranges of values are provided herein merely as examples.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Other systems, devices, methods, features and advantages of the subject matter described herein will be or will become apparent to one with skill in the art upon examination of the following figures and detailed description. It is intended that all such additional systems, devices, methods, features and advantages be included within this description, be within the scope of the subject matter described herein, and be protected by the accompanying claims. In no way should the features of the example embodiments be construed as limiting the appended claims, absent express recitation of those features in the claims.

We claim:

1. A method to deliver an aerosol of pirfenidone to treat a patient suffering from interstitial lung disease comprising:
(1) introducing an aqueous solution of pirfenidone in a medicine reservoir cup of a nebulizer;
(2) closing the medicine cup reservoir by attaching a closure to an opening in a housing of the nebulizer through which the aqueous solution was introduced to create a headspace within the medicine cup reservoir above the aqueous solution of pirfenidone;
(3) activating an aerosol generator having a vibrating mesh membrane to generate a pirfenidone aerosol formed from the aqueous pirfenidone solution in an aerosol mixing chamber, wherein activating the vibrating mesh nebulizer is coincident with inhalation of the pirfenidone aerosol through a mouthpiece opening in the aerosol mixing chamber by the patient suffering from the interstitial lung disease—wherein ambient pressure is maintained in the headspace of the medicine cup reservoir during activation of the aerosol generator by allowing air to enter the medicine cup reservoir through a vent pathway in the nebulizer;
(4) delivering the pirfenidone aerosol to the patient by inhalation, wherein the respirable delivered dose rate for the range of concentrations of the pirfenidone solution is between greater than 0.9 mg/min for the 4.0 mg/ml solution and greater than 4.3 mg/min for the 19.0 mg/ml pirfenidone solution to deliver a respirable dose of at least 7 mg of pirfenidone; and
(5) achieving a reduced decline in a baseline of forced vital capacity in the patient suffering from interstitial lung disease.

2. The method of claim 1, wherein the step of activating the aerosol generator generates an aerosol dose of pirfenidone in the aerosol mixing chamber, wherein the aerosol mixing chamber has a volume greater than 49 cubic centimeters.

3. The method of claim 1, wherein the step of delivering the pirfenidone aerosol is comprised of converting between 0.5 and 10 mLs of the aqueous pirfenidone solution to the pirfenidone aerosol.

4. The method of claim 1, wherein the activating step is comprised of the patient operating a control circuit that activates the vibrating mesh membrane for a duration that converts between 0.5 and 10 mLs of aqueous pirfenidone solution to the pirfenidone aerosol.

5. The method of claim 1, wherein the activating step is comprised of the patient operating a control circuit that activates the vibrating mesh membrane for a time interval determined by the volume of the aqueous pirfenidone solution in the medicine reservoir cup and a rate of delivery of the respirable delivered dose rate does not decrease during the duration of inhalation by the patient.

6. The method of claim 1, wherein the respirable delivered dose rate increases during the duration of inhalation by the patient.

7. The method of claim 1, wherein a daily dose level is greater than 25 mg of pirfenidone.

8. The method of claim 1, wherein the respirable delivered dose of the pirfenidone aerosol has a volumetric mean diameter (VMD) of about 2 μm to about 5 μm, a Geometric Standard Deviation (GSD) of emitted droplet size distribution of the pirfenidone aerosol of about 1.0 to about 3.4, and a fine particle fraction (FPF=% of aerosol particles ≤5 μm) of droplets emitted from the liquid nebulizer of at least about 45% greater than about 7 mg.

9. The method of claim 1, wherein a dose delivery rate is between greater than 2.8 and 6.25 mg of pirfenidone per minute.

10. The method of claim 1, wherein the nebulizer has output rate of at least 0.5 mL/min to convert the entire volume of the aqueous solution in medicine cup reservoir to the pirfenidone aerosol in between 1 and 20 minutes.

11. The method of claim 1, wherein the step of closing the medicine cup reservoir establishes a vent pathway between ambient air and the head space of the medicine cup reservoir.

12. The method of claim 11, wherein the vent pathway traverses the housing in the housing of the nebulizer and the closure seals the opening in the housing of the nebulizer through which the aqueous solution of pirfenidone was introduced.

13. The method of claim 11, wherein the vent pathway traverses the body of the enclosure.

14. The method of claim 11, wherein the delivering step is comprised of the patient activating the control circuit and inhaling the pirfenidone aerosol through a mouthpiece affixed to an end of the aerosol mixing chamber.

15. The method of claim 14, wherein the delivery step is further comprised of the intake of ambient air through a one way inspiratory valve in the aerosol mixing chamber during the inhalation by the patient.
